(19) United States
(12) Patent Application Publication
(10) Pub. No.: US 2019/0350648 A1
(43) Pub. Date: Nov. 21, 2019

(54) METHODS AND SYSTEMS FOR IN SITU EXCHANGE

(71) Applicant: Gynesonics, Inc., Redwood City, CA (US)

(72) Inventors: Christopher Michael OWENS, Huntington Beach, CA (US); Edmond Ming Wai CHIU, San Francisco, CA (US); Jiayu CHEN, Palo Alto, CA (US); Thomas Burnell REEVE, III, San Francisco, CA (US)

(21) Appl. No.: 16/414,040

(22) Filed: May 16, 2019

Related U.S. Application Data
(60) Provisional application No. 62/674,479, filed on May 21, 2018.

(51) Int. Cl. A61B 18/14; A61B 1/005; A61B 90/00

(52) U.S. Cl. CPC A61B 18/1492; A61B 1/04; A61B 90/361; A61B 1/0051

(57) ABSTRACT

An imaging component comprises shaft and a cavity extending across the shaft from its proximal end towards its distal end. The cavity removably receives at least one of a plurality of different instruments. A wall of the cavity comprises an elongated opening in communication with an exterior of the shaft at least partially along the shaft. An imaging transducer is coupled to the distal end of the shaft. The imaging component is advanced to a target site either alone or with a first instrument coupled thereto. A therapeutic or diagnostic procedure is performed with the first instrument. The first instrument is then retracted and removed from the imaging component while the imaging component stays at the target site. A second instrument is then coupled to the imaging component and advanced to the target site to perform a further therapeutic or diagnostic procedure.

### CROSS-REFERENCE TO RELATED APPLICATION(S)

The present application claims priority to U.S. Provisional Application No. 62/674,479 filed May 21, 2018, the full contents of which are incorporated herein by reference.

### BACKGROUND

The present disclosure relates to medical systems, devices, and methods. More particularly, the present disclosure relates to imaging components in use with therapeutic and diagnostic instruments.

Current systems, devices, and methods for imaging may be less than ideal in at least some respects. For example, many current devices may have limited flexibility for use in a variety of diagnostic and therapeutic procedures. For example, many current devices may not interface well with other therapeutic or diagnostic instruments. For example, many current devices may be expensive and/or difficult to clean. For example, many current devices may risk injuring a patient during insertion and/or removal.

Additionally or alternatively, current systems, devices, and methods for diagnosing and providing therapy may be less than ideal in at least some other respects. For example, in procedures where more than one instrument may be required, multiple instruments may need to be inserted or removed from a patient lumen, and these additional steps of insertion and removal may increase injury risk for the patient. Additionally or alternatively, many current methods may require removal of an imaging component many times during a single procedure, and the removal of the imaging component may limit the ability to continually and steadily view the surgical field during the procedure.

In light of the above, improved systems, devices, and methods for imaging a surgical field are desired. Such systems, devices, and methods would address at least some of the drawbacks above and would, for example, be less expensive, easier to clean, and/or able to be used for a greater variety of therapeutic and diagnostic procedures.

### SUMMARY

The present disclosure relates to imaging components in use with therapeutic and diagnostic instruments. In particular, the imaging components disclosed herein may be positioned in situ to capture images of the surgical field while a variety of therapeutic and/or diagnostic instruments may be exchanged at least partially through the imaging component. The imaging components disclosed herein may be used alone, in combination with only one instrument, or with multiple instruments. An exemplary imaging component may comprise a shaft and a cavity extending across the shaft from its proximal end towards its distal end. The cavity may removably receive at least one of a plurality of different instruments. A wall of the cavity may comprise an elongated opening in communication with an exterior of the shaft at least partially along the shaft. An imaging transducer may be coupled to the distal end of the shaft to continually image the surgical field when the imaging component is positioned in situ. The imaging component may be advanced to a target site either alone for imaging or with a first instrument coupled thereto. A first instrument may be inserted into the shaft of the imaging component in situ. A therapeutic or diagnostic procedure may be performed with the first instrument. The first instrument may then be retracted and removed from the imaging component. The imaging component may continually and steadily capture images of the surgical field before, during, and after retraction and removal of the first or other instrument(s). A second instrument can then be coupled to the imaging component and advanced to the target site to perform a further therapeutic or diagnostic procedure, without interrupting the imaging of the surgical field. The first instrument can be a diagnostic instrument to perform a diagnostic procedure, and the second instrument can be a therapeutic instrument to perform a therapeutic procedure as informed by the diagnostic procedure (or vice versa, or both the first and second instruments may be diagnostic instruments, or both the first and second instruments may be therapeutic instruments). Additional instruments may be coupled to the imaging transducer after removal and retraction of a second instrument. For example, a diagnostic procedure may be repeated in order to review therapeutic efficacy. In some cases, the imaging transducer may be used alone. In some cases, one or more disposable tubes may be coupled to the cavity to serve as a sterile, and optionally disposable, adapter for the different instruments to be coupled to and advanced along the imaging component.

Aspects of the present disclosure provide imaging components. An exemplary imaging component may comprise a shaft comprising a proximal end, a distal end, and a cavity extending across the shaft from the proximal end towards the distal end. The cavity may be configured to removably receive at least one of a plurality of different instruments. A wall of the cavity may comprise an elongated opening in communication with an exterior of the shaft at least partially along the shaft. The exemplary imaging component may further comprise an imaging transducer coupled to the distal end of the shaft.

The cavity may be defined by an exterior surface of the shaft. The exterior surface of the shaft may comprise only atraumatic edges. An edge of the elongated opening may be bent towards an interior of the cavity. The cavity may be configured to slidably receive the instrument. A distal portion of the cavity may be angled axially relative to the shaft. The distal portion of the cavity may be angled at about 3 to 45 degrees axially relative to the shaft.

At least one of the plurality of instruments may comprises a tube. The tube may be aligned to be in parallel with the shaft of the imaging component. The tube may be rotatable relative to the shaft while the shaft remains stationary. The tube may comprise a lumen configured to slidably receive a second instrument of the plurality of instruments. The tube may be configured to slidably receive the second instrument after the second instrument is aligned to be in parallel with the shaft of the imaging component. The second instrument may be rotatable relative to the shaft while the shaft remains stationary. The tube may be disposable. The second instrument may comprise a tissue collector. The tissue collector may comprise a biopsy needle. The second instrument may comprise a tissue ablation element. The tissue ablation element may comprise one or more of a radiofrequency (RF) ablation element, an ultrasonic ablation element, a heat-based ablation element, or a cryoablation element. The second instrument may comprise a resection tool. The second instrument may comprise an instrument for implantation of a device such as radiopaque markers, drug-eluting wireform, fertility/contraception treatment, anchoring system, herniation mesh, stent, or other devices. The second instrument may comprise instrumentation for providing detailed mapping of anatomy such as laser, x-ray, secondary ultrasound, or other devices. The first and second instruments may be any diagnostic or therapeutic device or may be a tube for receiving additional instruments.

At least one of the plurality of different instruments comprises a therapeutic or diagnostic instrument. The therapeutic or diagnostic instrument may comprise a tissue collector, a biopsy needle, a tissue ablation element, an optical scope, implantation device, and/or therapy electrodes. The tissue ablation element may comprise one or more of a radiofrequency (RF) ablation element, an ultrasonic ablation element, a heat-based ablation element, or a cryoablation element.

The shaft may be flexible. The shaft may be controllably flexed along a longitudinal axis thereof via a flex mechanism.

The imaging transducer may comprise an ultrasound transducer. The imaging transducer may comprise a light emitting diode (LED) or a camera.

The cavity may define a circular cross sectional area. The cavity may comprise a substantially uniform cross sectional area along the shaft. The cavity may comprise an asymmetrical cross sectional area. The cavity may extend across the shaft from the proximal end to the distal end.

Aspects of the present disclosure may provide imaging systems. An exemplary imaging system may comprise any of the imaging components described herein and a disposable tube slidably received within the cavity of the imaging component. The system may further comprise a second instrument removably received within a lumen of the disposable tube. The second instrument may be a diagnostic or therapeutic instrument, a tissue collector, a biopsy needle, an optical scope, implantation device, and/or a tissue ablation element. The tissue ablation element comprises one or more of a radiofrequency (RF) ablation element, an ultrasonic ablation element, a heat-based ablation element, a cryoablation element, etc.

Aspects of the present disclosure may provide methods of performing therapy or diagnosis at a target site. In an exemplary method, any of the imaging components described herein may be inserted into the subject. With the imaging component in situ, at least one of the plurality of instruments may be inserted into the cavity towards the target site, therapy or diagnosis may be performed using the instrument(s) at the target site, and the instrument(s) may then be removed from the cavity.

At least one of the plurality of instruments comprises a tissue collector, a biopsy needle, and/or tissue ablation element. The tissue ablation element may comprise one or more of a radiofrequency (RF) ablation element, an ultrasonic ablation element, a heat-based ablation element, or a cryoablation element. The instrument(s) may comprise a therapeutic or diagnostic instrument such as an optical scope, implantation device, or therapy electrodes.

The exemplary method may comprise steps of inserting a second instrument into the cavity towards the target site, performing therapy or diagnosis using the second instrument at the target site, and removing the second instrument from the cavity. The second instrument may be different from the at least one of the plurality of instruments. The method may be performed in laparoscopic surgery, non-invasively, and/or in minimally invasive surgery.

The second instrument may comprise a tissue collector, a biopsy needle, and/or tissue ablation element. The tissue ablation element may comprise one or more of a radiofrequency (RF) ablation element, an ultrasonic ablation element, a heat-based ablation element, or a cryoablation element. The second instrument may comprise a therapeutic or diagnostic instrument such as an optical scope, implantation device, or therapy electrodes.

Aspects of the present disclosure may provide methods of performing image guided ablation therapy. In an exemplary method, any of the imaging components as described herein may be inserted into a subject. With the imaging component in situ, a biopsy needle may be inserted into the cavity, pathology samples may be collected using the biopsy needle, the biopsy needle may be removed from the cavity, radiofrequency (RF) ablation elements may be inserted into the cavity, tissue may be ablated using the RF ablation elements, the RF ablation elements may be removed from the cavity, an optical scope may be inserted into the cavity, completion of the image guided ablation therapy may be confirmed using the optical scope, and the optical scope may be removed from the cavity. The method may be performed in laparoscopic surgery, non-invasively, and/or in minimally invasive surgery.

Aspects of the present disclosure may provide methods of coupling instruments. An imaging component may be advanced to within a surgical space. The imaging component may comprise a shaft comprising a proximal end and a distal end. A first instrument may be coupled to the imaging component for use in the surgical space. The first instrument may be a therapeutic or diagnostic instrument. The first instrument may be uncoupled from the imaging component while the imaging component remains within the surgical space. A second instrument may be coupled to the imaging component for use in the surgical space while the imaging component remains within the surgical space. The second instrument may be a therapeutic or diagnostic instrument different from the first instrument. The imaging component may comprise an imaging transducer comprising an ultrasound transducer. The method may be performed in laparoscopic surgery, non-invasively, and/or in minimally invasive surgery.

The coupling of the first instrument may occur while the imaging component remains within the surgical space. Alternatively or in combination, the coupling the first instrument may occur while the imaging component is outside of the surgical space.

The method may further comprise steps of collecting a tissue sample from the surgical space with the first instrument and/or ablating a region within the surgical space with the second instrument.

The method may further comprise performing therapy or diagnosis with the first instrument. The second instrument may be selected based on data gathered from said performing therapy or diagnosis with the first instrument. A parameter of therapy or diagnosis performed with the second instrument may be adjusted based on data gathered from said performing therapy or diagnosis with the first instrument. The data gathered may comprise image data, and the parameter may be adjusted by adjusting an ablation zone for the second instrument.

The imaging component may further comprise a cavity extending across the shaft from the proximal end towards the distal end. A wall of the cavity may comprise an elongated opening in communication with an exterior of the shaft at least partially along the shaft. The cavity may be defined by an exterior surface of the shaft. The exterior surface of the shaft may comprise only atraumatic edges. An edge of the elongated opening may be bent towards an interior of the cavity. The cavity may be configured to slidably receive the first instrument or the second instrument. A distal portion of the cavity may be angled axially relative to the shaft. The distal portion of the cavity may be angled at about 3 to 45 degrees axially relative to the shaft. A tube may be advanced to within the cavity. The tube may be aligned to be in parallel with the shaft of the imaging component. The tube may be rotatable relative to the shaft while the shaft remains stationary. The tube may comprise a lumen configured to slidably receive the first instrument or the second instrument. The tube may be configured to slidably receive the first instrument or the second instrument after the first or second instrument is aligned to be in parallel with the shaft of the imaging component. The first or second instrument may be rotatable relative to the shaft while the shaft remains stationary. The tube may be disposable.

The first or second instrument may comprise a tissue collector, a biopsy needle, a tissue ablation element, an optical scope, implantation device, and/or therapy electrodes. The tissue ablation element may comprise one or more of a radiofrequency (RF) ablation element, an ultrasonic ablation element, a heat-based ablation element, or a cryoablation element.

The shaft may be flexible. The shaft may be controllably flexed along its longitudinal axis via a flex mechanism.

The imaging component may comprise an imaging transducer comprising a light emitting diode (LED) or a camera. The cavity may define a circular cross sectional area. The cavity may comprise a substantially uniform cross sectional area along the shaft. The cavity may comprise an asymmetrical cross sectional area. The cavity may extend across the shaft from the proximal end to the distal end.

The 1) imaging component and 2) the first instrument or the second instrument may be coupled axially. The 1) imaging component and 2) the first instrument or the second instrument may be coupled laterally. The 1) the imaging component and 2) the first instrument or second instrument may be coupled with aid of magnets or indents.

Aspects of the present disclosure provide systems for performing therapy and/or diagnosis at a target site within a patient. An exemplary system may comprise a first therapeutic or diagnostic instrument, a second therapeutic or diagnostic instrument different from the first therapeutic or diagnostic instrument, and an imaging component configured to be removably coupled to both the first and second therapeutic or diagnostic instruments, either simultaneously or individually. The imaging component may be configured to be deliverable to the target site within the patient both (i) separately from the first and second therapeutic or diagnostic instruments, and (ii) coupled with the first and/or second therapeutic or diagnostic instruments. The imaging component may be configured to be removably coupled to both the first and second therapeutic or diagnostic instruments, either simultaneously or individually, after the imaging component is delivered to the target site within the patient. The imaging device may be used alone, in combination with only one instrument, or with multiple instruments.

The first and second therapeutic or diagnostic instruments may comprise two of the following: a tissue collector, a tissue ablation element, an optical scope, or therapy electrodes. The tissue collector may comprise a biopsy needle. The tissue ablation element may comprise one or more of a radiofrequency (RF) ablation element, an ultrasonic ablation element, a heat-based ablation element, or a cryoablation element.

The imaging component may comprise a shaft comprising a proximal end, a distal end, and a cavity extending across the shaft from the proximal end towards the distal end. A wall of the cavity may comprise an elongated opening in communication with an exterior of the shaft at least partially along the shaft. The cavity may be defined by an exterior surface of the shaft. The exterior surface of the shaft may comprise only atraumatic edges. An edge of the elongated opening may be bent towards an interior of the cavity. The cavity may be configured to slidably receive the instrument. A distal portion of the cavity may be angled axially relative to the shaft. The distal portion of the cavity may be angled at about 3 to 45 degrees axially relative to the shaft.

The system may further comprise a tube. The tube may be aligned to be in parallel with the shaft of the imaging component. The tube may be rotatable relative to the shaft while the shaft remains stationary. The tube may comprise a lumen configured to slidably receive the first or second instrument. The tube may be configured to slidably receive the first or second instrument after the first or second instrument is aligned to be in parallel with the shaft of the imaging component. The first or second instrument may be rotatable relative to the shaft while the shaft remains stationary. The tube may be disposable.

The shaft of the imaging component may be flexible. The shaft may be controllably flexed along its longitudinal axis via a flex mechanism.

The imaging component may comprise an imaging transducer comprising a light emitting diode (LED) or a camera. The cavity may define a circular cross sectional area. The cavity may comprise a substantially uniform cross sectional area along the shaft. The cavity may comprise an asymmetrical cross sectional area. The cavity may extend across the shaft from the proximal end to the distal end. The imaging transducer may comprise an ultrasound transducer.

Aspects of the present disclosure provide methods of performing therapy or diagnosis at a target site. An imaging component may be advanced to the target site. The imaging component may comprise 1) a shaft comprising a proximal end, a distal end, and a cavity extending across the shaft from the proximal end towards the distal end, wherein a wall of the cavity comprises an elongated opening in communication with an exterior of the shaft at least partially along the shaft, and 2) an imaging transducer coupled to the distal end of the shaft. Therapy or diagnosis may be performed using a first instrument inserted into the cavity and advanced to the target site.

The method may further comprise a step of inserting the first instrument into the cavity before advancing the imaging component to the target site. The first instrument may be inserted into the cavity after advancing the imaging component to the target site. The first instrument may be removed from the cavity while the imaging component remains at the target site. A second instrument may be inserted into the cavity and the second instrument may be advanced to the target site. Therapy or diagnosis may be performed using the second instrument.

The cavity of the imaging component may be defined by an exterior surface of the shaft. The exterior surface of the shaft may comprise only atraumatic edges. An edge of the elongated opening may be bent towards an interior of the cavity. The cavity may be configured to slidably receive the instrument.

The distal portion of the cavity may be angled axially relative to the shaft. The distal portion of the cavity may be angled at about 3 to 45 degrees axially relative to the shaft.

The imaging component may further comprise a tube. The tube may be aligned to be in parallel with the shaft of the imaging component. The tube may be rotatable relative to the shaft while the shaft remains stationary. The tube may comprise a lumen configured to slidably receive the first instrument. The tube may be configured to slidably receive the first instrument after the second instrument is aligned to be in parallel with the shaft of the imaging component. The first instrument may be rotatable relative to the shaft while the shaft remains stationary. The tube may be disposable.

The first instrument may comprise a tissue collector. The tissue collector may comprise a biopsy needle. Alternatively or in combination, the first instrument may comprise a tissue ablation element. The tissue ablation element may comprise one or more of a radiofrequency (RF) ablation element, an ultrasonic ablation element, a heat-based ablation element, or a cryoablation element. The instrument may comprise an optical scope. The instrument may comprise therapy electrodes.

The shaft of the imaging component may be flexible. The shaft may be controllably flexed along its longitudinal axis via a flex mechanism.

The imaging transducer may comprise a light emitting diode (LED) or a camera.

The cavity may define a circular cross sectional area. The cavity may comprise a substantially uniform cross sectional area along the shaft. The cavity may comprise an asymmetrical cross sectional area. The cavity may extend across the shaft from the proximal end to the distal end. The imaging transducer may comprise an ultrasound transducer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows a perspective view of an imaging component, in accordance with some embodiments.

FIG. 1B shows a side, cross-sectional view of the imaging component of FIG. 1A, in accordance with some embodiments.

FIG. 1C shows a magnified, perspective view of a distal end of the imaging component of FIG. 1A comprising a cavity, in accordance with some embodiments.

FIG. 2A shows a magnified, perspective view of a distal end of the imaging component of FIG. 1A with a tissue collector instrument disposed within the shaft of the imaging component, in accordance with some embodiments.

FIG. 2B shows a side, cross-sectional view of the imaging component of FIG. 1A with a biopsy instrument disposed within the shaft of the imaging component, in accordance with some embodiments.

FIG. 2C shows a magnified, perspective view of a distal end of the imaging component of FIG. 1A with a radiofrequency ablation instrument disposed within the shaft of the imaging component, in accordance with some embodiments.

FIG. 2D shows a top view of the imaging component of FIG. 1A with a drug delivery instrument disposed within the shaft of the imaging component, in accordance with some embodiments.

FIG. 2E shows a side, cross-sectional view of the imaging component of FIG. 1A with a needle disposed within the shaft of the imaging component, in accordance with some embodiments.

FIG. 3A shows an assembly view of an imaging system comprising the imaging component of FIG. 1A and an optical scope instrument, in accordance with some embodiments.

FIG. 3B shows an assembly view of the imaging system of FIG. 3A illustrating an attachment mechanism of the system, in accordance with some embodiments.

FIG. 4 shows a magnified, perspective view of a shaft of the imaging component of FIG. 1A wherein the shaft of the imaging component is flexible, in accordance with some embodiments.

FIG. 5A illustrates a perspective view of a system for diagnosing and/or providing therapy, including an imaging component configured to be removably coupled to multiple therapeutic and/or diagnostic instruments, in accordance with some embodiments. FIG. 5A shows the imaging component and the therapeutic and/or diagnostic instrument being separated.

FIG. 5B illustrates a perspective view of the system of FIG. 5A, with the therapeutic and/or diagnostic instrument being in a ready position to be removably coupled to the imaging component, in accordance with some embodiments.

FIG. 5C illustrates a perspective view of the system of FIG. 5A, with the therapeutic and/or diagnostic instrument being removably coupled to the imaging component, in accordance with some embodiments.

FIG. 6 shows a schematic of an imaging system comprising a digital processing device and a display visible to a user, in accordance with some embodiments.

FIG. 7A shows a schematic of the imaging component of FIG. 1A positioned within a uterus to image tissue thereof, in accordance with some embodiments.

FIG. 7B shows a surgical field image captured as in FIG. 7A that would be visible on a display, showing safety and treatment boundaries, in accordance with some embodiments.

FIG. 7C shows a surgical field image combining both a virtual image showing safety and treatment boundaries and the physical presence of a treatment needle, in accordance with some embodiments.

FIG. 7D shows a surgical field image combining both a virtual image showing safety and treatment boundaries as well as the physical presence of treatment needle and tines, in accordance with some embodiments.

FIG. 8 is a flow chart showing an exemplary method of performing therapy or diagnosis at a target site, in accordance with some embodiments.

FIG. 9 is a flow chart showing an exemplary method of performing image guided ablation therapy, in accordance with some embodiments.

FIG. 10 illustrates a schematic of an exemplary digital processing device programmed or otherwise configured with an imaging component, in accordance with some embodiments.

FIG. 11A shows a side, cross-section view of an imaging component having a shaft with a circular cross-section, in accordance with some embodiments.

FIG. 11B shows a side, cross-sectional view of an imaging component with edges bent inward towards the interior of the cavity, in accordance with some embodiments.

FIG. 12A illustrates a system for diagnosing and/or providing therapy, which includes an imaging component configured to be removably coupled to multiple therapeutic and/or diagnostic instruments in situ, in accordance with some embodiments. FIG. 12A shows the imaging component in use separate from the therapeutic and/or diagnostic instrument.

FIG. 12B illustrates the system of FIG. 12A, with the therapeutic and/or diagnostic instrument in a ready position to be removably coupled to the imaging component in situ, in accordance with some embodiments.

FIG. 12C illustrates the system of FIG. 12A, with the therapeutic and/or diagnostic instrument and the imaging component being removably coupled to one another in situ to be able to perform therapeutic and/or diagnostic procedure(s) in situ, in accordance with some embodiments.

### DETAILED DESCRIPTION

Embodiments of the present disclosure provide an imaging component comprising a cavity extending across (e.g., along) the length of a shaft, wherein the cavity may be configured to removably receive at least one of a plurality of different instruments. In some embodiments, the cavity of the imaging component may be partially open to an exterior of the shaft. The imaging component may comprise an imaging transducer at the distal end of the shaft. Additionally, the shaft of the imaging component may be configured such that additional therapeutic and/or diagnostic instruments/attachments may be removed and/or received and/or inserted during a medical procedure without disturbing the imaging component. Additionally or alternatively, the imaging component may remain in situ while the therapeutic and/or diagnostic instrument is received and/or removed. In some embodiments, the imaging component may be used without an additional therapeutic and/or diagnostic instrument coupled thereto. In some embodiments, the imaging component may be inserted and/or removed from a patient lumen without the presence of a therapeutic and/or diagnostic instrument. Such an imaging component may be used during a medical procedure such as, for example, non-invasive, minimally invasive, and/or laproscopic surgery.

Embodiments of the present disclosure may improve upon existing methods for imaging and treating a lesion in a tissue tract for procedures where multiple instruments may be required to diagnose and/or provide therapy during a single procedure. For example, an imaging component may be used for diagnosis; then a biopsy attachment may be inserted for a pathology sample; then an ablation attachment may be inserted for ablating any lesions; and then a further attachment or instrument may be inserted to perform additional procedures such a deliver drugs, implants, and/or therapeutic and/or diagnostic agents. The imaging component of the present disclosure may facilitate the insertion and removal of medical instruments by providing a shaft with atraumatic edges and a cavity configured to receive a plurality of different instruments. Additionally or alternatively, the imaging component may be used independently of an additional instrument or attachment. In such embodiments, the edges of the cavity may be smooth or rounded such that the edges may not catch on the patient tissue when used alone.

The cavity of an imaging component may improve upon existing methods for imaging and treatment by providing a cavity of an imaging component which may be easier to clean than a component with a closed cavity or lumen. The cavity of an imaging component may improve on existing methods for imaging and treatment by facilitating manufacture of the imaging component. Embodiments of the present disclosure may lower treatment cost by providing an imaging component with a disposable tube. Embodiments of the present disclosure may lower treatment costs by providing a reusable imaging component with a cavity into which disposable instruments may be inserted. Embodiments of the imaging component may provide a shaft which aligns the instrument with the ultrasound image at all times. Embodiments of the present disclosure may accommodate various instruments with different sizes and shapes. Embodiments of the present disclosure may provide a scale or position information to assist insertion of an instrument.

The systems and methods of the present disclosure may be particularly useful in the treatment of fibroids in a patient uterus. The imaging component may be deployed transvaginally and transcervically into the uterus, or in other cases, laproscopically into and through an exterior of the uterus or other organ or tissue tract. The imaging component may be used in conjunction with an additional instrument such as a biopsy needle; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the additional instrument may be used to deliver drugs, implants, or other therapeutic agents to the tissue to be treated. Additionally or alternatively, the tissue ablation element may comprise embodiments or variations of the needle/tine assemblies of commonly assigned U.S. Pat. Nos. 8,206,300, 8,262,574, and 8,992,427, the contents of which are incorporated herein by references.

Embodiments of the present disclosure may improve upon at least some of the systems and methods of the commonly assigned references by providing a shaft of an imaging component with atraumatic edges to enable use of the imaging component alone. In some embodiments, embodiments of the present disclosure may improve upon the ability to remove and/or receive an additional instrument by providing an imaging system without an attachment mechanism located in at least the portion of the system to be positioned in situ. In such an embodiment, the imaging component shaft may be non-cylindrically symmetric (e.g., oval or rectangular in cross-section) in order to reference the rotation of the additional instrument relative to the imaging component shaft. In some embodiments, the present disclosure may additionally or alternatively provide a shaft of an imaging component with a small angled portion to minimize damage risk to a surface of an imaging transducer surface by an instrument. Additionally or alternatively, the imaging component may comprise a disposable tube inserted within the cavity to provide, among many possible purposes, a working channel for inserting additional instruments with different diameters and making the system easier to clean.

The imaging components described herein may be used in a surgical procedure to provide a real time image of a target structure to be treated, including projecting safety and treatment boundaries as described in commonly assigned U.S. Pat. Nos. 8,088,072 and 8,262,577, the contents of which are incorporated by reference. The imaging components described herein may be useful for both imaging and treating uterine fibroids as described in commonly assigned U.S. Pat. No. 7,918,795, which is incorporated herein by reference. Other commonly assigned patents and published applications describing probes useful for treating uterine fibroids which may be used with the imaging components described herein include U.S. Pat. Nos. 7,815,571, 7,874,986, 8,506,485, 9,357,977, and 9,517,047, which are incorporated herein by references. Additional, commonly assigned patent applications describing systems for establishing and adjusting displayed safety and treatment zone boundaries which may be used in conjunction with the imaging components described herein include: U.S. Pat. Pub. No. 2014/0073910 , U.S. Pat. No. 8,992,427, U.S. pat. app. Ser. No. 15/811,520, and P.C.T. App. No. US2017/060674, which are each incorporated herein by reference. Commonly assigned patent application P.C.T. App. No. PCT/US2017/060674, describes mapping and planning system which may be used in conjunction with the imaging components described herein, is also incorporated herein by reference.

In some embodiments, the systems and methods of the present disclosure may provide an imaging component to be used in a variety of diagnostic and therapeutic procedures. Some embodiments may provide methods and systems to perform therapy or diagnosis on a volume of tissue. A volume of tissue may comprise a patient organ. A patient organ or bodily cavity may comprise for example: muscles, tendons, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, testes, a prostate, a heart, an artery, a vein, a spleen, a gland, a brain, a spinal cord, a nerve, etc. Some embodiments provide systems and methods suitable for laparoscopic surgery. Some embodiments provide systems and methods suitable for non-invasive surgery. Some embodiments provide systems and methods suitable for minimally invasive surgery. Some embodiments provide systems and methods suitable for robotic or robot assisted surgery.

Reference will now be made in detail to various embodiments, examples of which are illustrated in the accompanying drawings. In the following detailed description, numerous specific details are set forth in order to provide a thorough understanding of the invention and the described embodiments. However, the invention is optionally practiced without these specific details. In other instances, well-known methods, procedures, components, and circuits have not been described in detail so as not to unnecessarily obscure aspects of the embodiments.

It will be understood that, although the terms “first,” “second,” etc. are optionally used herein to describe various elements, these elements should not be limited by these terms. These terms are only used to distinguish one element from another. For example, a first instrument could be termed an instrument sensor, and, similarly, a second instrument could be termed a first instrument, without changing the meaning of the description, so long as all occurrences of the “first instrument” are renamed consistently and all occurrences of the second instrument are renamed consistently. The first instrument and the second instrument are both instruments, but they are not the same instrument.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

For ease of explanation, the figures and corresponding description below may be described below with reference to uterine imaging, specifically, in conjunction with the diagnosis and ablation and/or treatment of uterine fibroids. However, one of skill in the art will recognize that a similar imaging component may be used with similar instruments in other therapeutic applications for example: instruments for tissue biopsy, for drug delivery, for fluid infusion and/or aspiration, and for the treatment of cancers, tumors, fibroids, and other masses, malignant or benign, in any suitable bodily lumen.

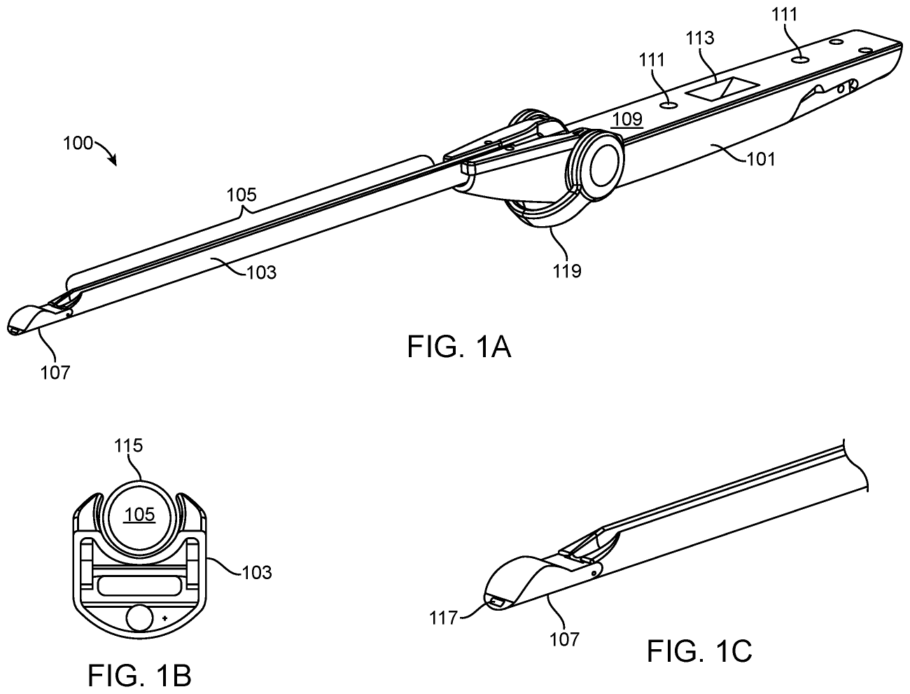

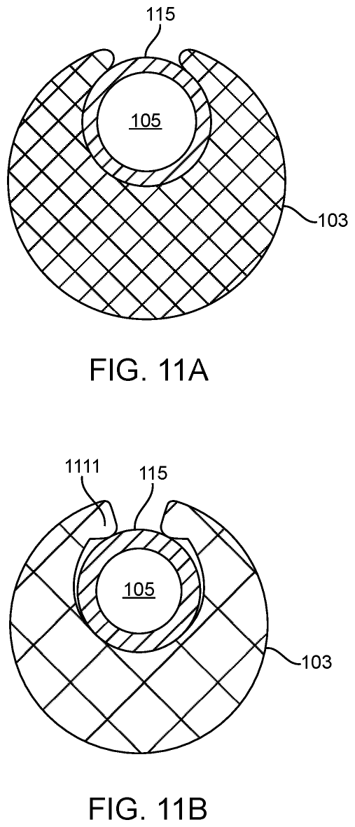

FIG. 1A shows an illustration of an imaging component 100, in accordance with some embodiments. Imaging component 100 may comprise a handle portion 101 connected to an imaging shaft 103. At the distal end of imaging shaft 103 may be coupled an imaging transducer 107. The imaging shaft may comprise a proximal end and a distal end with a cavity 105 extending across the length of the shaft from the proximal end towards the distal end. The cavity 105 may be at least partially open to the exterior of the shaft. For example, a side, or wall of the cavity may comprise an elongated opening in communication with the exterior of the shaft. The elongated opening may be in communication with the exterior of the shaft at least partially along the length of the shaft. In some embodiments, an edge of the elongated opening may be bent towards an interior of the cavity of the shaft (for example, see FIG. 11B further described below). The length of the shaft may be sufficiently long to fully access the uterus of a patient while the handle portion 101 remains exterior to the patient. Additionally or alternatively, the shaft may comprise a length significantly longer than the distance sufficient to fully access a patient uterus. The side opening may be open along the full length of the shaft or it may be open only partially along the length of the shaft. The side opening may be open, for example, for greater than three-fourths the length of the shaft, for greater than half the length of the shaft, or for greater than one quarter the length of the shaft. The cavity 105 may be configured to receive at least one of a plurality of different additional instruments or attachments, such that a first instrument may be received by the cavity, the first instrument may be removed from the cavity, and a second instrument may be received by the cavity.

The handle portion 101 may be one part of a two-part handle such that when a first instrument or a second instrument is received the two handle portions may combine to form a single handle. The inside of the handle portion 109 may comprise alignment elements 111 such that a first part and a second part may be reproducibly aligned with respect to one another after changing instruments. The alignment elements may be configured such that a first part and a second part may be sufficiently secured with respect to one another to use the two handle portions as a single handle. In some embodiments, the alignment elements may comprise magnets. I n other embodiments, alignment elements may comprise for example: latches, hooks, or any other mechanism suitable to removably combine a two-part handle. The handle portion may additionally comprise a positioning element 113, such as a slot to accommodate a complementary protrusion or other element on the opposite handle portion, in order to provide a more secure reference between parts of the two-part handle. The positioning element may comprise a mechanical feature to secure the instrument relative to the imaging component by limiting translation of the instrument on the axis of the shaft of the imaging component.

In other embodiments, imaging component 100 may be configured to be used with an instrument which does not have a handle portion. In such embodiments, the handle portion 101 of the imaging component 100 is sufficient to be used alone to guide the imaging component during a procedure. In some embodiments, imaging component 100 may have a scale or a guide on the inside of the handle portion 109 in order to gauge the insertion depth of an instrument. In other embodiments, the imaging component may be used without an instrument. In some embodiments, a scale may facilitate embodiments where the instrument does not have a handle. In other embodiments, a scale may facilitate the insertion of a component of the instrument in embodiments where the instrument has a handle.

FIG. 1B shows a cross-sectional view of an imaging component 100, in accordance with some embodiments. The body of the shaft may comprise internal structure in order to carry electronics or other associated components to control the imaging transducer. The shaft may also comprise a wire system or other flex mechanism in order to allow the shaft to controllably bend, flex, or deflect the distal end of the shaft. The shaft may comprise a channel or duct to direct fluid (e.g., water, saline, etc.) to a distal end of the shaft and onto a tissue surface. Imaging shaft 103 may be round in cross-section or take a shape with sufficiently softened, chamfered, rounded, or beveled edges such that the edges may be atraumatic to a patient opening during insertion or removal of an imaging component with or without an instrument. Shaft 103 may additionally comprise a smooth exterior surface. Shaft 103 may be made of a material such that the surface may be deformable to allow the shaft to bend or adapt to the shape of a bodily lumen.

The cavity 105 of imaging shaft 103 may be configured to slidably receive one or more of a plurality of instruments. In some embodiments, the cavity may be defined by an exterior surface of the shaft. In some embodiments, the cavity may be partially open along a wall, such that the cavity may be in communication with the exterior of the shaft. The opening may be sufficiently closed to provide structural support such that when the imaging component may be inserted into a patient bodily lumen, the opening of the lumen may not be significantly disturbed by the insertion or removal of an instrument. Optionally, the exterior surface of the shaft may comprise only atraumatic edges. The cavity 105 of imaging shaft 103 may be sufficiently open such that when instruments of different sizes may be received or inserted into the cavity, the cavity may allow some distortion of the cavity opening. The cavity may facilitate cleaning of the imaging component.

FIG. 11A shows a cross-section view of an imaging component having a shaft with a circular cross-section, in accordance with some embodiments. The imaging component of FIG. 11A may be sufficiently circular in cross-section such that the imaging component may be rotated without disturbing a patient lumen. FIG. 11B shows a cross-sectional view of an imaging component with edges bent inward towards the interior of the cavity, in accordance with some embodiments. The inward bent edges 1111 of a cavity may serve to support the opening of a bodily lumen such that the shaft may be inserted or removed atraumatically from a bodily lumen with or without an instrument.

While the cavity of the shaft in the illustrated example may define a circular cross sectional area, in other embodiments the cavity may be elliptical or any other geometric shape with sufficiently softened, rounded, or beveled edges and corners such that insertion or removal of the shaft may not damage the patient bodily lumen. In some embodiments, the cavity may be non-cylindrically symmetric. In some embodiments, the cavity may be asymmetrical to provide an axis for alignment of the instrument within. The cavity may be open for less than three-quarters its perimeter in cross-section, additionally or alternatively, the cavity may be open for less than half its perimeter, less than a quarter its perimeter, and less than one eighth its perimeter. In other embodiments, the cavity of the shaft of the imaging component may be closed to the exterior of the shaft, and an instrument may be slidably inserted fully interior to the shaft of the imaging component.

In some embodiments, the cavity may comprise a substantially uniform cross sectional area along the shaft. In other embodiments, a portion of the length of the shaft may have a different cross section than another portion of the length of the shaft. In an example, the proximal portion of the shaft may be asymmetric to provide an axis for alignment of an instrument and the distal portion of the shaft may have a circular cross sectional area. In another embodiment, the cavity tapers toward the end of the shaft. In such an example, the taper may facilitate feeding an instrument into the cavity. In some embodiments, the cross sectional area of the cavity may narrow in diameter to allow greater flexibility of the distal end of the shaft.

In some embodiments, imaging shaft 103 may additionally comprise a tube 115 to be positioned at the cavity 105 of imaging shaft 103. Tube 115 may comprise a lumen. The lumen of tube 115 may be configured to slidably receive one or more of a plurality of instruments. Tube 115 may be aligned in parallel with the shaft of the imaging component, such that an additional instrument/attachment may be slidably received by the tube. Subsequently, the tube 115 may slidably receive the additional instrument/attachment after it has been aligned to be in parallel with the shaft of the imaging component. In some embodiments, the tube 115 may be disposable. In some embodiments, the tube 115 may be reusable such as by being un-coupled from the imaging shaft 103, washed, and autoclaved. Tube 115 may have an exterior surface wherein the surface is substantially in contact with the inner wall of cavity 105. Tube 115 may have an interior surface of a different geometry to the outer surface configured to receive one or more of a plurality of instruments. In some embodiments, a second tube may be removably inserted into the first tube and the second tube may have a different inner lumen geometry than the first, thereby aiding in the insertion of one or more of a plurality of instruments. In some embodiments, the tube 115 may be rotated relative to the imaging component. In some embodiments, the tube 115 may fully rotate relative to the imaging component in either direction under the control of a user within the shaft of the imaging component. In some embodiments, the tube 115 may be internally or externally lubricated to facilitate insertion or removal of an instrument.

The tube 115 may be inserted into the bodily lumen in situ with the imaging component yet advanced therein. Additionally or alternatively, the tube 115 may be inserted into the shaft of the imaging component prior to insertion of the imaging component into the bodily lumen. The tube 115 may have sufficient structural integrity to support a bodily lumen during insertion of the imaging component without an instrument. When an additional instrument is inserted into the tube 115 or the tube 115 is inserted into the imaging component in situ, disruption to the bodily lumen may be minimized. The tube 115 may be made of a material that can be sterilized. The tube 115 may be made of a material that may be of low enough cost that it may be disposed of after a single use. Exemplary materials for a disposable tube may comprise polyimide, PTFE, Urethanes and thermoplastics like Pebax or Nylon, etc. Tube 115 may be made of a material comprising sufficient elasticity in order to adapt to an instrument of a size somewhat larger or smaller than the perimeter of the tube. In embodiments where the cavity is not circular, the tube may take the shape of the cavity or it may take another shape.

The tube 115 may lower treatment costs by facilitating insertion and/or removal of an additional instrument into the cavity of the imaging component 100 and thereby preventing damage to the surface of the cavity 105 of the imaging component 100. The tube 115 may lower cost by facilitating cleaning of the cavity 105 of the imaging component 100. The tube 115 may lower cost of treatment by providing an inexpensive component which may act as an adapter for a variety of different therapeutic and/or diagnostic instruments/attachments, such as being provided in a variety of different inner geometries suitable for the different instruments/attachments but having a uniform outer geometry to be removably coupled to the same single imaging component 100. For example, a disposable tube with a smaller inner diameter may facilitate the insertion and control of a needle with a smaller outer diameter than the inner diameter of the shaft of the imaging component.

FIG. 1C shows a magnified view of a distal end of the imaging component comprising a cavity, in accordance with some embodiments. The distal end of the imaging component may comprise an imaging transducer 107. The imaging transducer may comprise an ultrasound transducer and/or a plurality of ultrasound transducers. The ultrasound transducer may operate at a frequency of 500 kHz, 1 MHz, 5 MHz, 10 MHz, 20 MHz, 100 MHz, or a range defined by any two of the preceding values. Some embodiments of the ultrasound transducer may comprise specifications of other transducers from the commonly assigned references incorporated herein.

In some embodiments, the distal end of the imaging transducer 117 may additionally comprise a light emitting diode and/or a camera in order to provide images to a user. In such embodiments, the imaging component may serve as an optical scope as well as an ultrasound imaging platform. The distal end of the imaging transducer may comprise optical components, such as an optic fiber, a relay lens, an objective lens, etc.

The imaging transducer 107 may be configured to be deflectable. The imaging transducer may be configured to deflect relative to the longitudinal axis of the shaft of the imaging component. In some embodiments, the distal end of an imaging component comprises a hinge to facilitate deflection of an imaging transducer. The deflection of the imaging transducer may be controlled by a deflection lever 119 on the handle portion 101 of the imaging component. The one or a plurality of imaging transducers may be oriented by the deflection of the imaging transducer. The one or a plurality of imaging transducers may be oriented by the deflection of the imaging transducer in order to facilitate maintaining the field of view of an image during a treatment. Additionally or alternatively, the ultrasound transducers may be aligned radially and/or axially to image multiple views simultaneously. Deflection of the imaging transducer may be induced in order to avoid obstruction of an instrument. Additionally or alternatively, deflection of the imaging transducer may be used to deflect a flexible instrument within the cavity. The distal end of the shaft may comprise an interlock system, similar to those in the incorporated references, in order to prevent the imaging transducer from obstructing an instrument or being damaged by sharp edges of an instrument. Actuation of the deflection lever may function in a manner similar to that described in U.S. Pat. No. 8,992,427, incorporated herein by reference. The deflection lever 119 may deflect the imaging transducer by less than 45 degrees and additionally or alternatively, for example, less than 120 degrees, less than 90 degrees, less than 60 degrees, less than 30 degrees, less than 15 degrees, and less than 5 degrees.

The distal end of the imaging component may comprise atraumatic edges in order to facilitate insertion of the imaging component with or without an instrument in the cavity. The distal end of the cavity of the imaging component may additionally or alternatively comprise a portion angled axially relative to the shaft, such that a distal end of an instrument may be deflected upward as it is pushed out the distal end of the cavity. The distal end of the cavity of the imaging component may comprise an angled portion with an angle of 3 to 45 degrees. The distal end of the cavity of the imaging component may comprise an angled portion with an angle at less than 45 degrees and additionally or alternatively, for example, less than 90 degrees, less than 60 degrees, less than 30 degrees, less than 15 degrees, and less than 5 degrees.

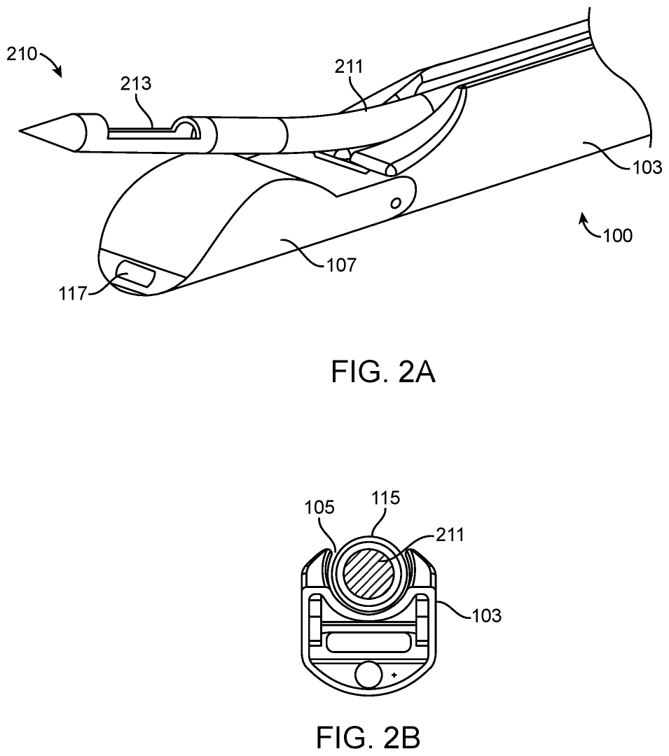

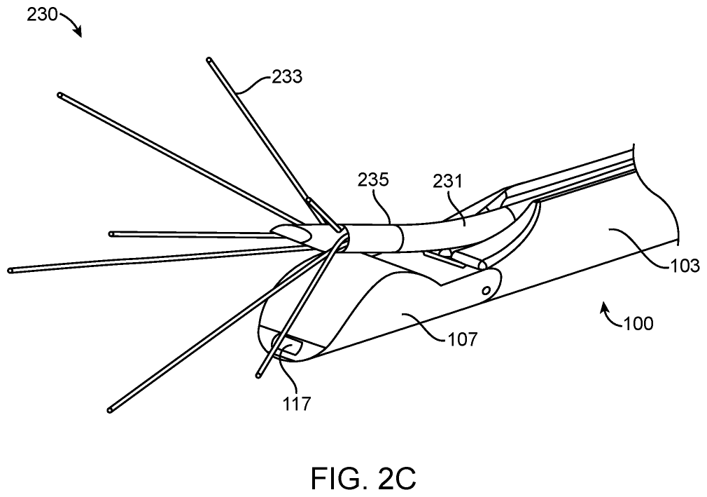

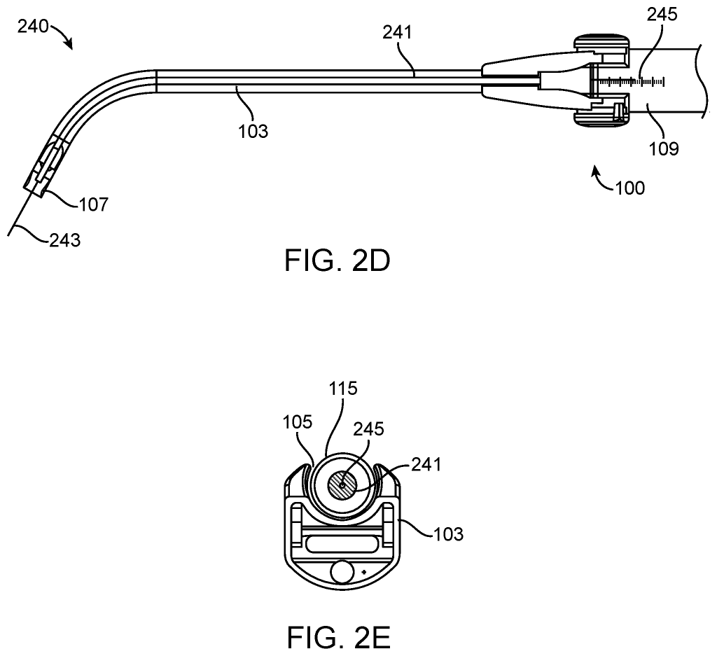

The cavity of the imaging component may be configured to slidably receive one or more of a plurality of instruments. In some embodiments, the imaging component may be configured to receive one or a plurality of therapeutic or diagnostic instruments. In some embodiments, at least one of the plurality of different instrument may be a therapeutic or diagnostic instrument. In some embodiments, the instrument may comprise an instrument such as a biopsy needle; an optical scope; implantation device; therapy electrodes; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. FIGS. 2A-2E show instruments which may be slidably received by the imaging component. One of ordinary skill in the art will recognize that many instruments, including those disclosed in the following figures, may be used with the imaging component disclosed herein.

FIG. 2A shows a magnified view of a distal end of the imaging component with a tissue collector instrument 210 disposed within the shaft 105 of the imaging component 100, in accordance with some embodiments. The tissue collector element may be used to extract tissue and/or cell pathology samples for examination by a medical professional to determine the extent of a disease. In some embodiments, the tissue collector may comprise a biopsy needle. Tissue collector 210 may comprise a shaft of a tissue collector 211, which has a distal end and a proximal end. The shaft of tissue collector 211 may be configured to detach from a handle component of the instrument or may be configured to be used without a handle component such that the tissue collector 210 may be disposable.

The shaft of tissue collector 211 may be made of a pliable and/or flexible material such that it may be deflected by the imaging transducer and/or an angled portion within the cavity of the shaft. In the illustrated example, a distal end of a shaft of a tissue collector is deflected upward by an angled portion within the cavity of the shaft. The distal end of a shaft of a tissue collector may be deflected up in order to avoid damage of the imaging transducer, among other possible purposes. The distal end of the cavity of the imaging component may comprise a portion angled axially relative to the shaft, such that a distal end of an instrument may be deflected upward as it is pushed out the distal end of the cavity. The distal end of the cavity of the imaging component may comprise an angled portion angled at less than 45 degrees and additionally or alternatively, for example, less than 90 degrees, less than 60 degrees, less than 30 degrees, less than 15 degrees, and less than 5 degrees.

Additionally or alternatively, the shaft of the imaging collector may comprise a wire system or other means to deflect the distal end of the tissue collector such that a distal end of a tissue collector does not damage the imaging transducer. The distal end of the tissue collector instrument may comprise a slot or opening 213 into which tissue may be collected. In some embodiments, the tissue collector may rotate relative to the shaft. In some embodiments, the tissue collector may fully rotate relative to the shaft in either direction under the control of a user within the shaft of the imaging component while the shaft remains stationary, such that the slot 213 may scrape, scoop, or otherwise collect tissue.

The shaft of the tissue collector may be longer than the shaft of the imaging transducer such that the slot or opening may collect tissue from deep inside the uterus or other body cavity. In some embodiments, the shaft of the tissue collector may be two inches longer than the shaft of the imaging transducer. Additionally or alternatively for example, the shaft may be six inches longer, may be four inches longer, may be two inches longer, may be the same length, or may be within a range of any two of the preceding values.

FIG. 2B shows a cross-sectional view of an imaging component with a tissue collector instrument 211 disposed within the shaft of the imaging component, in accordance with some embodiments. Tissue collector 211 may be disposed within a tube 115 disposed within the cavity 105 of the imaging component. Additionally or alternatively, tissue collector 211 may be disposed within the cavity of the imaging component without the use of a tube. While the shaft of the collector instrument in the illustrated example may be circular, in other embodiments, the shaft of the collector instrument may be elliptical any other geometric shape such that the shaft may be inserted or removed from the cavity of the imaging component. In some embodiments, the shaft of the collector may be asymmetrical to provide an axis for alignment of the instrument within the cavity of the imaging component. In some embodiments, the cavity comprises a substantially uniform cross sectional area along the length of the shaft. In other embodiments, the cross sectional area changes along the length of the shaft such as, for example, the proximal end of the shaft may be asymmetric to provide an axis for alignment while the distal end of the shaft may be circular.

FIG. 2C shows a magnified view of a distal end of the imaging component with an ablation instrument 230 disposed within the shaft of the imaging component, in accordance with some embodiments. The ablation instrument 230 may contain a needle assembly comprising needle 235 and, optionally, tines 233. The shaft of the ablation instrument 231 may be deployed from the shaft of an imaging component 103. Additionally or alternatively, the needle may be deployed from a lumen of a tube 115. The ablation instrument may comprise one or more of, for example, a radiofrequency (RF) ablation element, an ultrasonic ablation element, a heat-based ablation element, a cryoablation element, and any other type of ablation elements known to one of ordinary skill in the art.

Ablation instrument 230 may be disposed within a tube 115 disposed within the cavity 105 of the imaging component. Additionally or alternatively, ablation instrument 230 may be disposed within the cavity of the imaging component without the use of a tube. While the shaft of the ablation instrument 231 in the illustrated example may be circular, in other embodiments, the shaft of the ablation instrument may be elliptical or any other geometric shape such that the shaft may be inserted or removed from the cavity of the imaging component. In some embodiments, the shaft of the ablation instrument may be asymmetrical to provide an axis for alignment of the instrument within the cavity of the imaging component.

The shaft of the ablation instrument 231 may be made of a pliable and/or flexible material such that it may be deflected by the imaging transducer and/or an angled portion within the cavity of the shaft. Additionally or alternatively, the shaft of the ablation instrument may comprise a wire system or other means to deflect the distal end of the ablation instrument such that a distal end of the ablation instrument does not damage the imaging transducer. In some embodiments, the ablation element may rotate relative to the imaging component. In some embodiments, the ablation instrument may fully rotate relative to the imaging component in either direction under the control of a user within the shaft of the imaging component while the shaft remains stationary, such that the tines may be optimally aligned.

The needle assembly may be constructed and controlled by a user, for example, as previously described in commonly owned U.S. Pat. Nos. 8,206,300, 8,262,574, and 8,992,427, the full disclosures of which are incorporated herein by reference. The needle assembly may be integrated into an instrument handle such that the position and deployment of the needle and tines may be controlled by the user. The handle may be constructed, for example, as previously described in commonly owned U.S. Pat. No. 8,992,427, the full disclosure of which is incorporated herein by reference. The needle assembly may be compatible with systems and methods for improved safety and treatment boundaries during the treatment of uterine fibroids as, for example, described in the incorporated references.

FIG. 2D shows a view of an imaging component with a drug delivery instrument 240 disposed within the shaft 105 of the imaging component 100, in accordance with some embodiments. A drug delivery instrument may serve as a platform to inject therapeutic agents into the tissue of a patient. Exemplary therapeutic agents may comprise analgesics, anesthetics, hemostatics, antibiotics, steroids, anticoagulants, anti-inflammatories, etc. Additionally or alternatively, the drug delivery instrument may be configured to deliver to target tissue, one or more drug eluting, drug releasing, or otherwise therapeutic and/or diagnostic seeds, pellets, or other implants. The drug delivery instrument may comprise needle 243 disposed inside a distal end of a shaft of a drug delivery instrument 241. The shaft of a drug delivery instrument 241 may comprise a distal end and a proximal end. The shaft of the drug delivery instrument may be longer than the shaft of the imaging transducer such that the needle may inject agents deep inside the uterus. In some embodiments, the shaft of the drug delivery instrument may be two inches longer than the shaft of the imaging transducer. Additionally or alternatively, for example, the shaft may be six inches longer, may be four inches longer, may be two inches longer, may be the same length, or may be within a range of any two of the preceding values.

The shaft of drug delivery instrument 241 may be made of a pliable and/or flexible material such that it may be deflected by the imaging transducer and/or an angled portion within the cavity of the shaft. Additionally or alternatively, the shaft of the drug delivery instrument may comprise a wire system or other means to deflect the distal end of the drug delivery instrument such that a distal end of a drug delivery instrument does not damage the imaging transducer. In some embodiments, the drug delivery instrument may rotate relative to an imaging component. In some embodiments, the drug delivery instrument may fully rotate relative to an imaging component in either direction under the control of a user within the shaft of the imaging component while the shaft remains stationary.

The shaft of a drug delivery instrument may be detachable from a handle component of the instrument or maybe constructed without a handle component such that the drug delivery instrument may be disposable. In the illustrated embodiment, drug delivery instrument 240 does not have a handle portion. In such embodiments, the handle portion 101 of the imaging component 100 may be used to guide the drug delivery instrument during a procedure. Shown in FIG. 2D imaging component 100 may have a scale, a guide, or other indicia 245 on the inside face of the handle portion 109 in order to gauge the insertion depth of a needle 243 of a drug delivery instrument 240.

FIG. 2E shows a cross-sectional view of the imaging component with a needle disposed within the shaft of the imaging component 103, in accordance with some embodiments. The shaft of a drug delivery instrument 241 comprising a needle 243 may be disposed within a tube 115 disposed within the cavity 105 of the imaging component. Additionally or alternatively, the shaft of a drug delivery instrument 241 may be disposed within the cavity of the imaging component without the use of a tube. While shaft of the drug delivery instrument in the illustrated example may be circular, in other embodiments the shaft of the drug delivery instrument may be elliptical any other geometric shape such that the shaft may be inserted or removed from the cavity of the imaging component. In some embodiments, the shaft of the drug delivery instrument may be asymmetrical to provide an axis for alignment of the instrument within the cavity of the imaging component. In some embodiments, the drug delivery instrument may rotate relative to the imaging component. In other embodiments, the drug delivery instrument may fully rotate relative to the imaging component in either direction under the control of a user within the tube of the shaft of the imaging component while the shaft remains stationary.

FIGS. 2A to 2E illustrate exemplary instruments which may be disposed within the shaft of an imaging component, which examples are not intended to be limiting. Other examples may comprise a fluid infusion and/or aspiration instrument. A fluid infusion and/or aspiration instrument may comprise an instrument with a shaft comprising a lumen therein, configured to conduct a fluid to a tissue of a patient. A fluid infusion and/or aspiration instrument may deliver fluid to cool a tissue. Additionally or alternatively, a fluid infusion and/or aspiration instrument may deliver a fluid to clean a tissue. Additionally or alternatively, a fluid infusion and/or aspiration instrument may deliver a fluid to inflate a bodily cavity. A fluid infusion and/or aspiration instrument may deliver a solution and/or suspension comprising a therapeutic agent, such as an antiseptic, an anesthetic, an analgesic, an antibiotic, a steroid, etc. A fluid infusion and/or aspiration element may be integrated into any of the instruments described herein. Alternatively, a fluid infusion and/or aspiration element may comprise an instrument to be inserted and retracted as a step during a multi-instrument procedure.

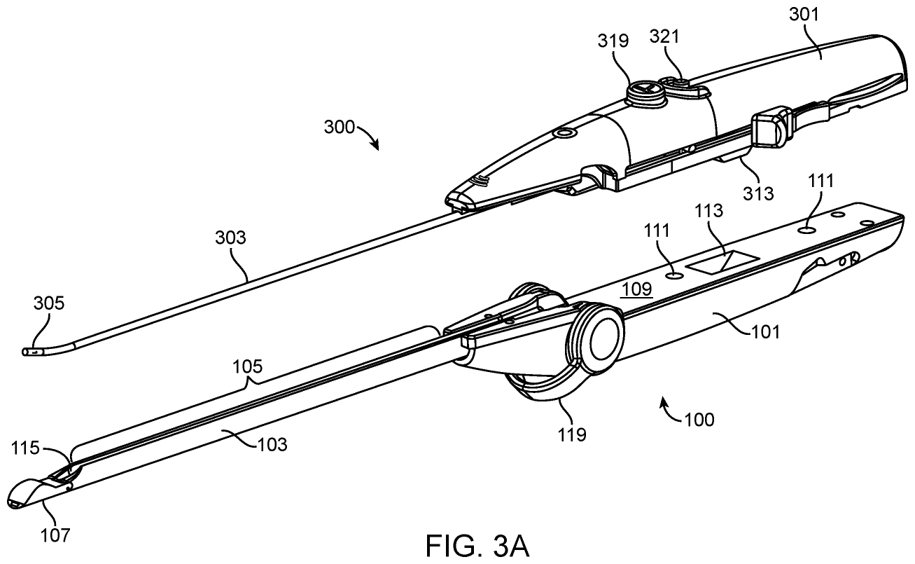

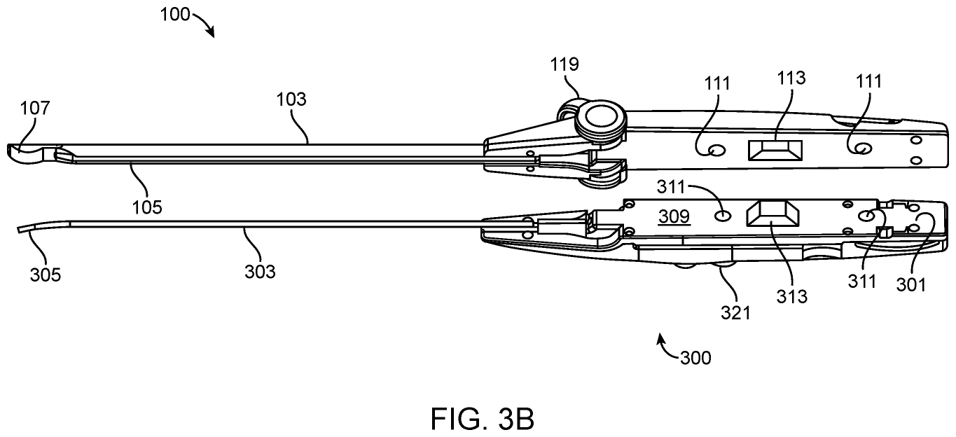

FIG. 3A shows an assembly view of an imaging system comprising an imaging component 100 and an optical scope instrument 300, in accordance with some embodiments. While an optical scope element may be shown in the illustrated embodiment, optical scope instrument 300 may be any other suitable instrument, for example, any of the instruments disclosed herein. Illustrated in FIG. 3A, the imaging system may slidably receive a disposable tube 115 within the cavity 105 of the imaging component. In some embodiments, the imaging system may comprise a disposable tube slidably received within the cavity of the imaging component. In such embodiments, an instrument may be removably received with a lumen of the disposable tube. Additionally or alternatively, the cavity of the imaging component may be configured to slidably receive one or more of a plurality of instruments, which instruments may comprise various therapeutic and/or diagnostic instruments.

In illustrative examples, the imaging component may removably receive an instrument such as a biopsy needle; a tissue collector instrument; an optical scope; implantation device; therapy electrodes; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. Additionally or alternatively, with or without the use of a disposable tube, the imaging component may removably receive any of the instruments illustrated in FIG. 2A-2E.

In the illustrated embodiment, the distal end 305 of the optical scope instrument may comprise a light emitting diode and/or a camera in order to provide images to a user. In such embodiments, the optical scope instrument may serve as an endoscope. The distal end 305 of the optical scope element may comprise optical components, such as an optic fiber, a relay lens, an objective lens, etc. The optical scope instrument 300 may comprise a shaft of an optical scope instrument 303, which has a distal end and a proximal end. The shaft of optical scope instrument 303 may be configured to detach from a handle component of the instrument or may be configured to be used without a handle component such that the optical scope instrument 300 may be disposable.

The shaft of optical scope instrument 303 may be made of a pliable and/or flexible material such that it may be deflected by the imaging transducer and/or an angled portion within the cavity of the shaft. Additionally or alternatively, the shaft of the optical scope instrument may comprise a (e.g., push, pull, and/or rotate/torque) wire system or other means to deflect the distal end of the optical scope instrument. Deflection of a distal end of an optical scope instrument may serve to prevent damage the imaging transducer and/or allow multiple image angles may be collected. In some embodiments, the optical scope element may rotate relative to the imaging component. In some embodiments, the optical scope instrument may fully rotate relative to the imaging component in either direction under the control of a user within the shaft of the imaging component while the shaft remains stationary, such that multiple image angles may be collected.

The shaft of the optical scope instrument may be longer than the shaft of the imaging transducer such images may be collected from deep inside the uterus. In some embodiments, the shaft of the optical scope instrument may be two inches longer than the shaft of the imaging transducer. Additionally or alternatively, for example, the shaft may be six inches longer, may be four inches longer, may be two inches longer, may be the same length, or may be within a range of any two of the preceding values.

In the illustrated embodiment, the optical scope instrument comprises a handle portion 301. While a handle portion 301 may be shown connected to an optical scope in the illustrated example similar handle portions may be connected to any suitable instrument, such as those disclosed herein. The handle portion 301 may be the second part of a two-part handle such that when an optical scope instrument may be slideably inserted into the imaging component the two handle portions may combine to form a single handle. The handle portion may additionally comprise a positioning element 313, in order to provide a more secure reference between parts of the two-part handle. Positioning element 313 may mate with slot 113. In such embodiments, the handle portion may comprise a release control 321, which may be actuated by a user, to retract the positioning element into the handle and allow the two-handle to be separated.

The handle portion may additionally comprise one or a plurality of control elements 319. Control elements 319 may allow a medical professional to control the distal end of an instrument. In one example, the control element controls a wire system which may reproducibly deflect or steer a distal end of an instrument. Additionally or alternatively, the control element may rotate a shaft of an instrument with the cavity of the imaging component or within the disposable tube. In another example, the control element scoops tissue in a tissue collector instrument. In another example, the control element deploys a needle assembly comprising optional tines in an ablation instrument. Additionally or alternatively, the control element begins the ablation procedure. In another example, the control element applies pressure to inject a chemical though a drug delivery instrument. In another example, the control element begins or ends image collection in an optical scope instrument.

FIG. 3B shows an assembly view of an imaging system illustrating an attachment mechanism of a system, in accordance with some embodiments. The inside of the handle portion 309 may comprise alignment elements 311. Alignment elements 311 may be configured such that the optical scope instrument may be reproducibly aligned with respect the imaging component after changing instruments. Additionally or alternatively, the alignment elements may sufficiently secure the instrument and the imaging component with respect to one another to use the two handle portions as a single handle. In some embodiments, the alignment elements may comprise magnets. In other embodiments, the alignment elements may comprise for example: latches, hooks, or any other mechanism suitable to removably combine a two-part handle. The inside of the handle portion 309 may additionally comprise a positioning element 313, in order to provide a more secure reference between parts of the two-part handle. In such embodiments, the handle portion may comprise a release control 321, which may be actuated by a user, to retract the positioning element into the handle and allow the two-handle to be separated.

In some embodiments, a method of detecting or sensing the identification of removable instruments is provided when coupling the imaging component and the removable instrument. The imaging component may include software to recognize the removable instrument and manage the interconnection between the imaging component and removable instrument. The sensor or mechanism may be, by way of non-limiting examples, optical, RF, magnetic, biometric, electronic and mechanical IDs and readers. The method will ensure only qualified removable devices are received on the imaging device to ensure that only compatible devices may be used with the imaging component.

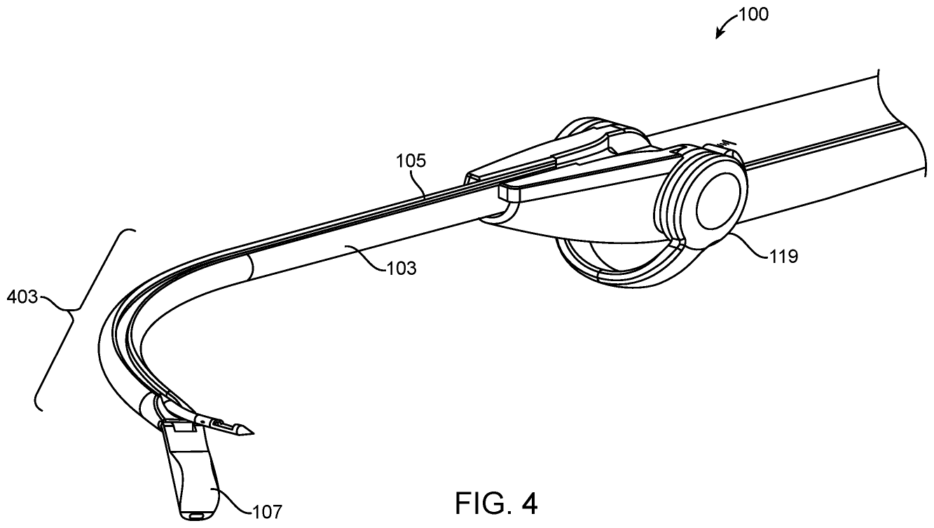

FIG. 4 illustrates a shaft of an imaging component wherein the shaft of the imaging component may be flexible, in accordance with some embodiments. In the illustrated embodiment, the shaft of the imaging component may comprise a flexible shaft portion 403. The body of the flexible portion of the shaft may comprise internal structure in order to carry electronics or other associated components to control the imaging transducer. The imaging transducer may comprise a channel or duct to direct fluid (e.g., water, saline, etc.) to a distal end of the shaft and onto a tissue surface. The flexible portion may comprise a fraction of the length of the shaft of the imaging component. In some embodiments, the flexible portion comprises less than three-quarters the length of the shaft. Additionally or alternatively, the flexible portion may comprise less than a quarter the length of the shaft, and less than one eighth the length of the shaft, and the full length of the shaft.

The cross-section of the flexible portion of the shaft may continue the geometry of the shaft such that no gaps or traumatic edges may be created between the flexible portion of the shaft and the shaft. The flexible portion may be round in cross-section or take a shape with sufficiently softened, chamfered, rounded or beveled edges such that the edges may be atraumatic to a patient opening during insertion or removal of an imaging component with or without an instrument. The flexible portion may additionally comprise a smooth exterior surface. The flexible portion may be made of a material such that the surface may be deformable to allow the flexible portion to bend or adapt to the shape of a bodily lumen.

The cavity of the flexible portion may be configured to slidably receive one or more of a plurality of instruments. The cavity of the flexible shaft portion may be configured to continue the shape of the cavity of the shaft such that no gaps or traumatic edges may be created between the flexible portion of the shaft and the shaft. In some embodiments, the cavity of the flexible portion may be partially open along a wall, such that a lumen of the cavity of the flexible portion may be in communication with the exterior of the shaft. The opening of the flexible portion may be sufficiently closed to provide structural support such that when the imaging component may be inserted into a patient bodily lumen, the opening of the lumen may not be significantly disturbed by the insertion or removal of an instrument. In some embodiments, the edges of a cavity of the flexible portion may bend inward towards the interior of the cavity, such as in the embodiment illustrated in FIG. 11B. The inward bent edges of a cavity of the flexible portion may serve to support the opening of a bodily lumen such that the shaft may be inserted or removed atraumatically from a bodily lumen with or without an instrument. The cavity of the flexible portion may be sufficiently open such that when instruments of different sizes may be received or inserted into the cavity, some distortion of the cavity opening may occur. The cavity may facilitate cleaning of the imaging component by providing access to the interior of the cavity from its exterior.

While the cavity of the flexible portion in the illustrated example defines a circular cross sectional area, in other embodiments the cavity of the flexible portion may be elliptical any other geometric shape with sufficiently softened, rounded, or beveled edges and corners such that insertion or removal of the shaft of the flexible portion does not damage the patient bodily lumen. In some embodiments, the cavity of the flexible portion may be asymmetrical to provide an axis for alignment of the instrument within. The cavity of the flexible portion may be open for less than three-quarters of its perimeter in cross-section, additionally or alternative, the cavity may be open for less than half its perimeter, less than a quarter its perimeter, and less than one eighth its perimeter. In other embodiments, the cavity of the flexible portion of the shaft of the flexible portion may be closed to the exterior of the shaft of the flexible portion, and an instrument may be slidably inserted fully interior to the shaft of the flexible portion.

In some embodiments, the flexible shaft portion may be constructed from a pliable and/or flexible material such that it may be flexed within a patient bodily lumen. In some embodiments, the shaft may be controllably flexed along its longitudinal axis via a flex mechanism. Additionally or alternatively, the flexible portion of the shaft may comprise a wire system or other flex mechanism in order to allow the flexible portion to controllably bend, flex, or deflect the distal end of the flexible portion. The flex mechanism may be controlled by a control element on a handle portion of the imaging component.

In the illustrated example, the flexible portion may be flexed axially to about a 90 degree angle with respect to the handle. Additionally or alternatively, the flexible portion may be flexed axially to, for example, less than 180 degrees, less than 120 degrees, less than 90 degrees, less than 45 degrees, less than 10 degrees, less than 1 degrees. Additionally or alternatively, the flexible portion may be flexed in an anterior-posterior axis relative to the handle of the imaging component. In some embodiments, the flexible portion may be flexed in an anterior-posterior axis to, for example, less than 180 degrees, less than 120 degrees, less than 90 degrees, less than 45 degrees, less than 10 degrees, less than 1 degrees. Additionally or alternatively, the flexible portion may be flexed in a medial-lateral axis relative the handle of the imaging component. In some embodiments, the flexible portion may be flexed in a medial-lateral axis to, for example, less than 180 degrees, less than 120 degrees, less than 90 degrees, less than 45 degrees, less than 10 degrees, less than 1 degrees.

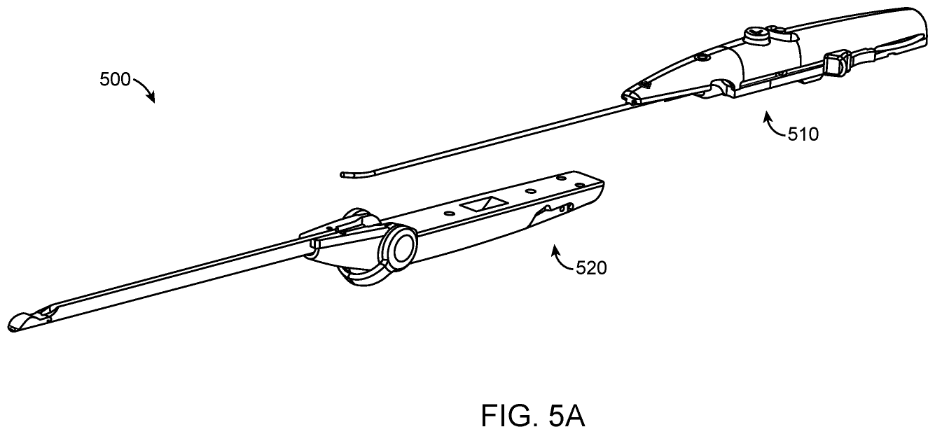

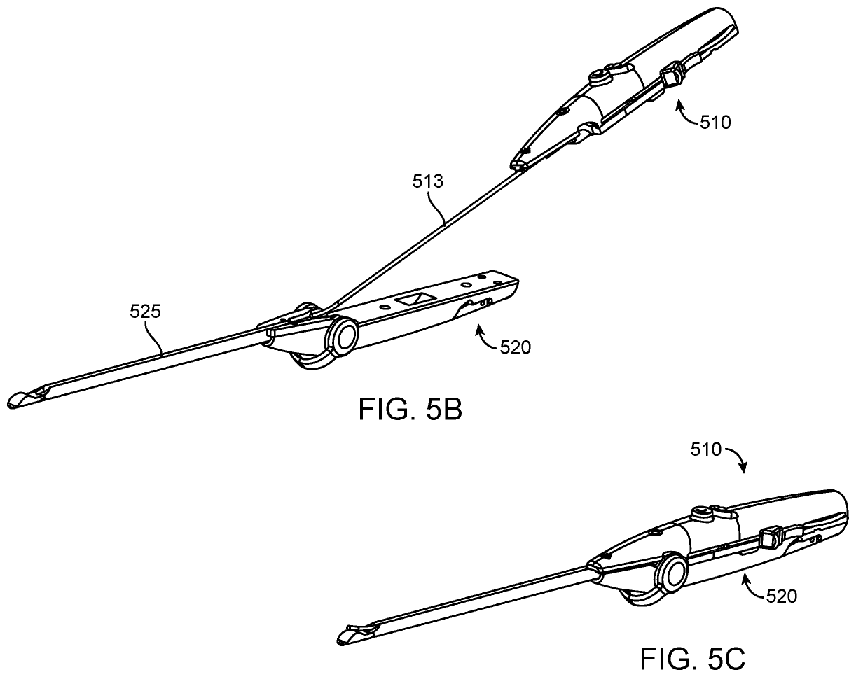

FIG. 5A illustrates a system for diagnosing and/or providing therapy, which may be removably coupled to multiple therapeutic and/or diagnostic instruments, in accordance with some embodiments. A system for performing therapy and/or diagnosis may comprise a therapeutic or diagnostic instrument 510 and an imaging component 520.An instrument 510 of the system for performing therapy and/or diagnosis may comprise a therapeutic or diagnostic instrument, such as, for example, any of the therapeutic or diagnostic instruments described herein. In some embodiments, the imaging component may be used in conjunction with an instrument such as a biopsy needle; a tissue collector, an optical scope; implantation device; therapy electrodes; a tissue ablation element, such as for example a radiofrequency ablation element, an ultrasonic ablation element, a heat-based ablation element, a cryoablation element, etc.; and/or any other instrument suitable to be disposed within the cavity of the imaging component. Additionally or alternatively, the instrument may be used to deliver drugs or other therapeutic agents to the tissue to be treated. FIGS. 2A-2E shows exemplary instruments which may be slidably received by the imaging component. In some embodiments, the system may comprise a first and a second therapeutic or diagnostic instrument. An imaging component 520 may comprise an imaging component, such as, for example, examples, embodiments, and variations on the imaging component described herein.

FIG. 5B illustrates a system for diagnosing and/or providing therapy with a therapeutic and/or diagnostic instrument being removably coupled to an imaging component, in accordance with some embodiments. As shown, the instrument 510 may be axially aligned with respect to the imaging component 520. Additionally, the distal end of the shaft of the instrument 513 may be fed into the proximal end of the cavity 525 of the imaging component. Subsequently, the instrument may be advanced toward imaging component, such that the shaft of the instrument is slidably received by cavity of the imaging component. The instrument may be slidably removed from the imaging component by a similar procedure.

FIG. 5C illustrates a system for diagnosing and/or providing therapy with a therapeutic and/or diagnostic instrument removably coupled to an imaging component, in accordance with some embodiments. The system for diagnosing therapy may comprise retention elements such as hooks, latches, or the mechanical features described herein in order to secure the instrument 510 to the imaging component 520. The system for diagnosing and/or providing therapy may be configured to couple to a plurality of instruments. For example, a first instrument may be coupled to an imaging component, and, subsequently, a second instrument may be coupled. The imaging component may be configured to be coupled to both the first and second therapeutic and/or diagnostic instrument either simultaneously or individually. For example, if the first instrument is a disposable tube, the second instrument may be slidably inserted within the first instrument. In some embodiments, the imaging component may be configured to be deliverable to the target site within the patient previously coupled with the first and/or second therapeutic or diagnostic instruments exterior to the target site. Additionally or alternatively, the imaging component may be configured to be removably coupled to both the first and second therapeutic or diagnostic instruments, either simultaneously or individually, after the imaging component is delivered to the target site within the patient (e.g., the instrument may be coupled in situ).

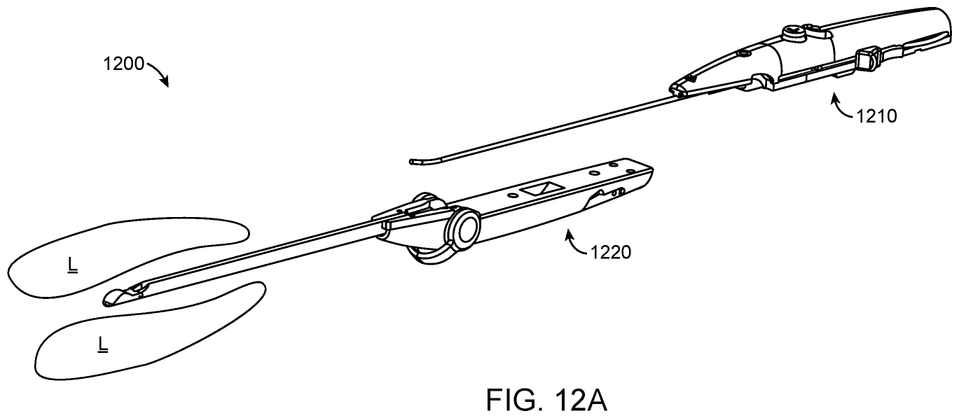

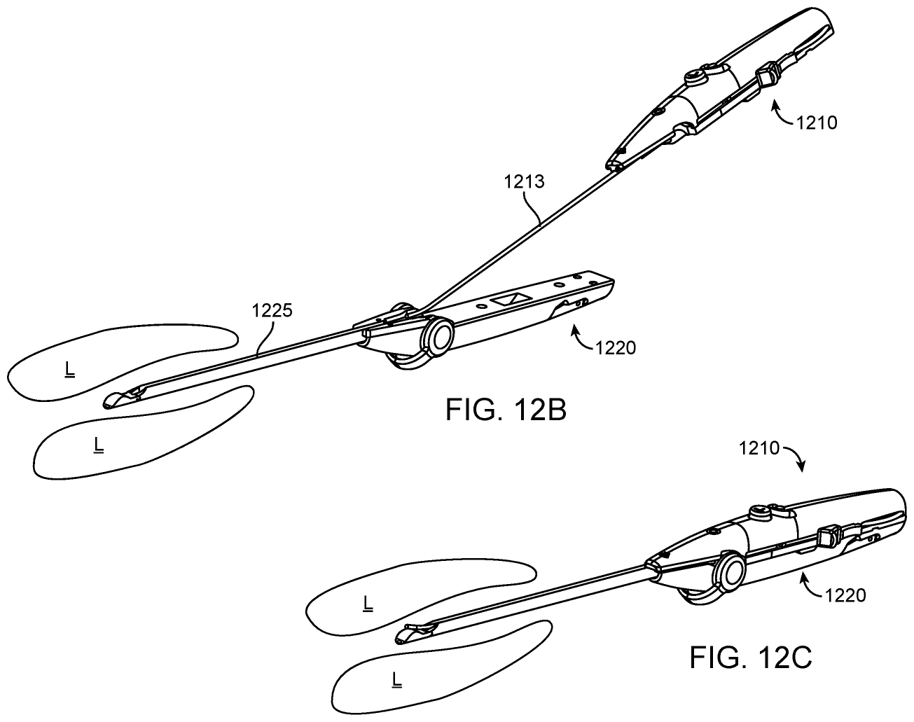

FIG. 12A illustrates a system for diagnosing and/or providing therapy, which may be removably coupled to multiple therapeutic and/or diagnostic instruments in situ, in accordance with some embodiments. FIG. 12A shows the imaging component in use separate from the therapeutic and/or diagnostic instrument, in accordance with some embodiments. A system for performing therapy and/or diagnosis may comprise a therapeutic or diagnostic instrument 1210 and an imaging component 1220.An instrument 1210 of the system for performing therapy and/or diagnosis may comprise a therapeutic or diagnostic instrument, such as, for example, any of the therapeutic or diagnostic instruments described herein. In some embodiments, the imaging component may be used in conjunction with an instrument such as a biopsy needle; a tissue collector, an optical scope; implantation device; therapy electrodes; a tissue ablation element, such as for example a radiofrequency ablation element, an ultrasonic ablation element, a heat-based ablation element, a cryoablation element, etc.; and/or any other instrument suitable to be disposed within the cavity of the imaging component. Additionally or alternatively, the instrument may be used to deliver drugs or other therapeutic agents to the tissue to be treated. FIGS. 2A-2E show exemplary instruments which may be slidably received by the imaging component. In some embodiments, the system may comprise a first and a second therapeutic or diagnostic instrument. An imaging component 1220 may comprise an imaging component, such as, for example, examples, embodiments, and variations on the imaging component described herein. As shown the illustrated embodiment, imaging component 1220 may disposed within a bodily lumen L of a patient without an additional therapeutic and/or diagnostic instrument positioned within the shaft of the imaging component. In some examples, the imaging component 1220 may be used without a therapeutic and/or diagnostic instrument.

FIG. 12B illustrates a system for diagnosing and/or providing therapy with a therapeutic and/or diagnostic instrument being removably coupled to an imaging component in situ, in accordance with some embodiments. As shown, the instrument 1210 may be axially aligned with respect to the imaging component 1220, while the imaging component is disposed within the patient lumen. Additionally, the distal end of the shaft of the instrument 1213 may be fed into the proximal end of the cavity 1225 of the imaging component, while the imaging component remains in situ. Subsequently, the instrument may be advanced toward imaging component, such that the shaft of the instrument is slidably received by cavity of the imaging component in situ. The instrument may be slidably removed from the imaging component by a similar procedure. Instrument 1210 may be slidably inserted without displacing the distal end of the imaging component. Instrument 1210 may be slidably inserted without discontinuation or disruption of the imaging functionality of imaging component 1220.

FIG. 12C illustrates a system for diagnosing and/or providing therapy with a therapeutic and/or diagnostic instrument removably coupled to an imaging component in situ, in accordance with some embodiments. The system for diagnosing therapy may comprise retention elements such as hooks, latches, or the mechanical features described herein in order to secure the instrument 1210 to the imaging component 1220. The system for diagnosing and/or providing therapy may be configured to couple to a plurality of instruments. For example, a first instrument may be coupled to an imaging component, and, subsequently, a second instrument may be coupled. The imaging component may be configured to be coupled to both the first and second therapeutic and/or diagnostic instrument either simultaneously or individually. For example, if the first instrument is a disposable tube, the second instrument may be slidably inserted within the first instrument.

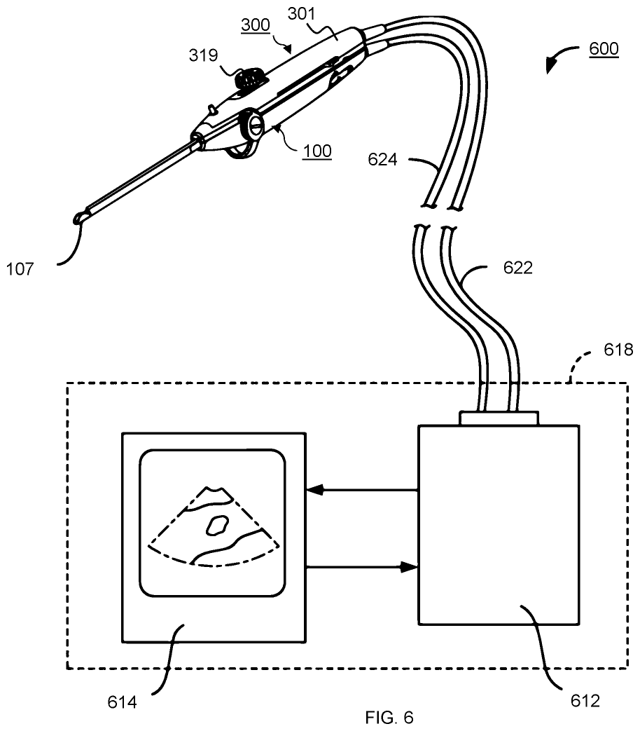

FIG. 6 shows an imaging system 600 comprising a digital processing device 612 and a display visible to a user 614, in accordance with some embodiments. As illustrated in FIG. 6, an imaging system 600 may additionally comprise an imaging component 100 and an instrument 300. The digital processing device 612 may comprise one or more processors configured with instructions to set and record both treatment parameters and imaging parameters. The display 614 may be included in a common enclosure 618; however, in other embodiments, the display 614 may be remote to a digital processing device and/or the imaging component 100. The imaging component 100 may be connected to the digital processing device 612 by an imaging cord 624 to provide the image(s) captured by to the digital processing device 612 to be displayed by the display 614; however, additionally or alternatively, the imaging component may communicate with the digital processing device wirelessly. The instrument 300 may be connected to the digital processing device 612 by an instrument cord 622; however, additionally or alternatively, the instrument may communicate with the digital processing device wirelessly. In embodiments where the imaging component and the instrument are connected by cords, the digital processing device may supply power to both components.

The instrument 300 may comprise a handle portion 301 having a slidably mounted control elements 319 on its upper surface. In some embodiments, the control elements 319 may control the positioning of internal stops within the handle which may monitored by the processor 612 in order to calculate the size and position of the boundaries of the targeting region and/or the safety region which are shown on the display 614. In embodiments where instrument 300 is an ablation element, the stops may also serve to physically limit deployment of the needle and optionally tines.

Some embodiments of the methods and systems of the present disclosure may be integrated with systems and methods for establishing and adjusting displayed safety and treatment zone boundaries. Such embodiments may include systems and methods of the incorporated references including: U.S. Pat. Pub. No. 2014/0073910, US. Pat. No. 8,992,427, U.S. pat. app. Ser. No. 15/811,520, and P.C.T. App. No. US2017/060674, the contents of which are incorporated herein by reference. Some embodiments of the methods and systems of the present disclosure may be integrated with systems and methods for mapping and planning systems. Such embodiments may include systems and methods of the incorporated references including P.C.T. App. No. PCT/US2017/060674.

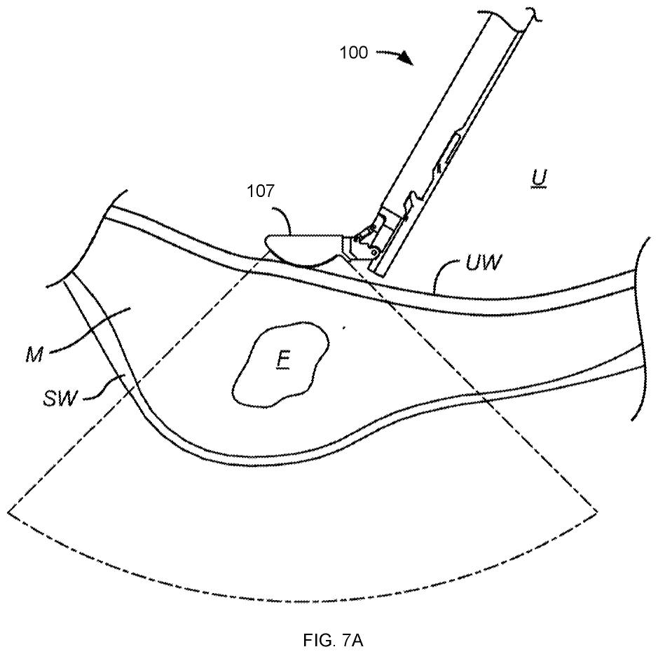

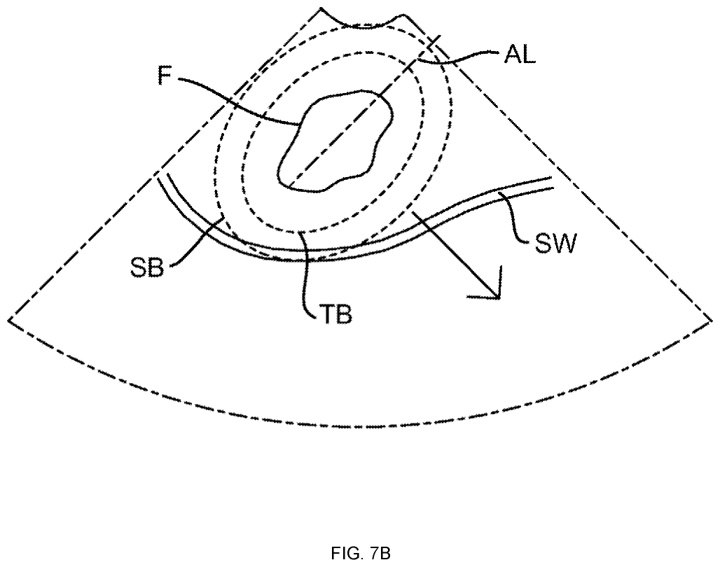

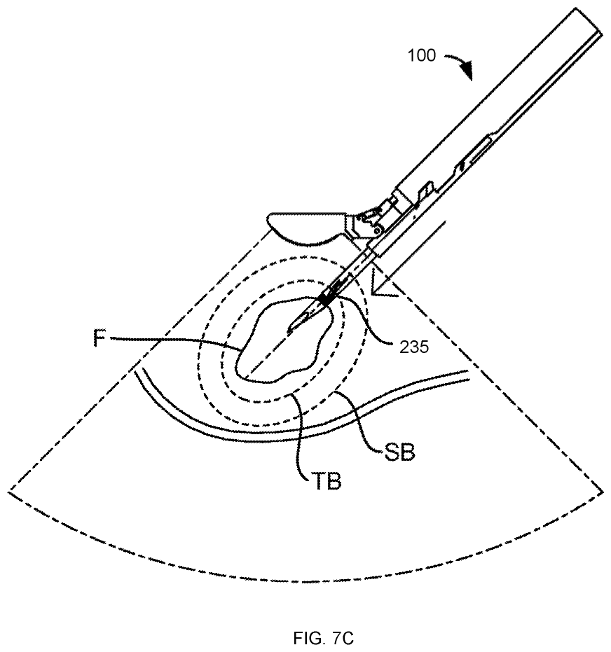

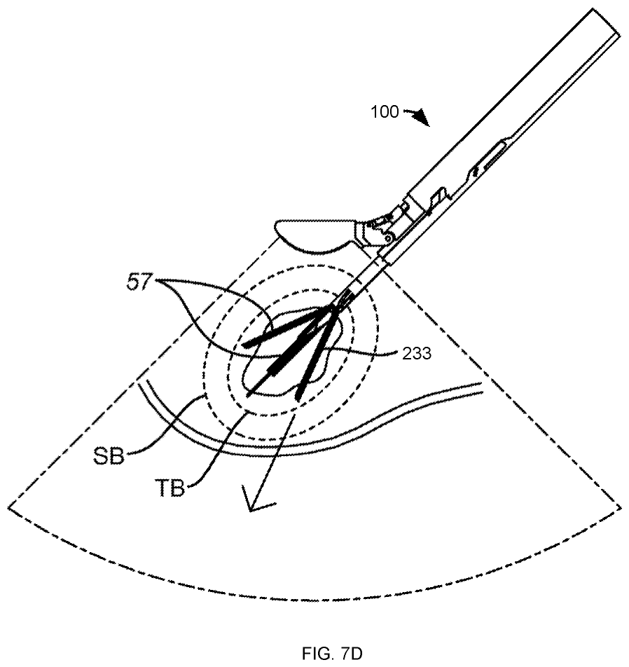

FIG. 7A illustrates an imaging component which may be used to treat a fibroid F located in the myometrium M in a uterus U beneath a uterine wall UW (the endometrium) and surrounded by the serosal wall SW. The imaging component 100 can be introduced transvaginally and transcervically (or alternately laparoscopically) to the uterus, and the imaging transducer 107 deployed to image the fibroid within a field of view indicated by the broken lines.

FIG. 7B shows an image that would be visible on a display, showing safety and treatment boundaries, in accordance with some embodiments. In some embodiments, once the fibroid is located on the display 614, the controls on the handle may be used to locate and size both a treatment boundary TB and a safety boundary SB. In some embodiments, initially, the virtual boundary lines TB and SB may neither be positioned over the fibroid nor properly sized to treat the fibroid. Prior to beginning therapy, the physician may want to both position and size the boundaries TB and SB for proper treatment. As the imaging transducer 107 may be already positioned against the uterine wall UW the only way to advance the treatment and safety boundaries may be to move the boundaries forward by actuating the control element 319. In some embodiments, this may cause the treatment and safety boundaries TB and SB to move forwardly along the axis line AL and thereby translate the area to be treated. This may cause the virtual boundaries on the real-time image display 614 to move over the image of the fibroid. Additionally or alternatively, the size of the treatment boundary TB may be enlarged or shrunk in order to mitigate the risk of affecting healthy and/or more sensitive tissue around the area of treatment.

In embodiments where the instrument is a tissue ablation element, while holding imaging component 100 steady, the physician may then advance a needle slide, causing the needle 235 to extend into the fibroid F, as shown in FIG. 7C. The illustration in FIG. 7C includes a representation of the imaging component 100, which corresponds to the physical probe which is present in the patient. The remainder of FIG. 7C corresponds to the image present on the target display 614.

After needle 235 has been fully deployed as limited by an optional physical or virtual needle stop housing in the instrument handle 301, the tines 233 may be deployed by advancing a tine slide a target level of tine deployment is reached as indicated by engagement of the tine slide with an optional tine stop or visually on the display. Optionally, the imaging component 100 may be rotated about a central axis (typically aligned with the axis of the needle 235) to confirm the treatment and safety boundaries in all planes of view about the fibroid. Display 614 will show the position of the treatment and safety boundaries in real time relative to the target fibroid and serosa. The tines are then configured as shown in FIG. 7D, and power can be supplied to the tines (and optionally the needle) in order to achieve treatment within the boundary depicted by the virtual treatment boundary TB. Again, FIG. 7D mixes both the virtual image which would be present on the display 614 as well as the physical presence of the imaging component 100.

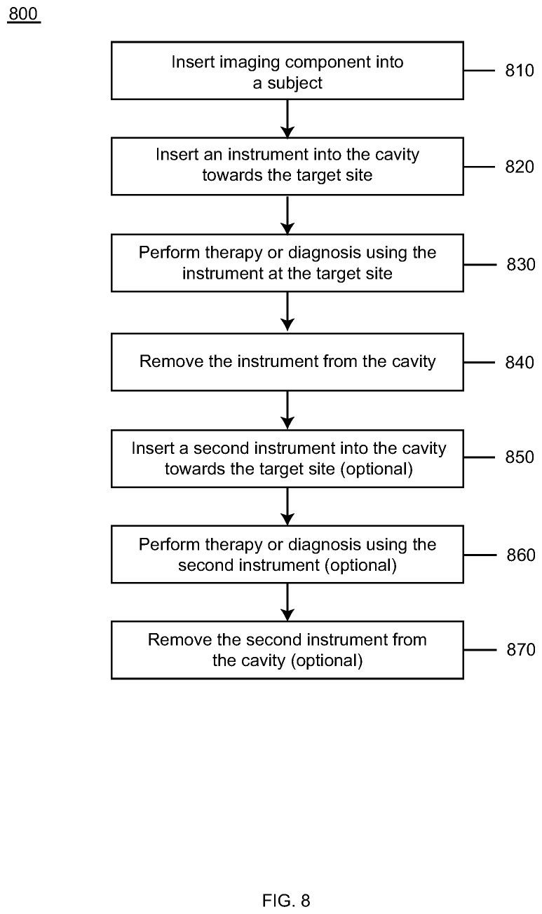

Embodiments of the present disclosure may provide a method of performing therapy or diagnosis at a target site. FIG. 8 shows an exemplary method 800 of performing therapy or diagnosis at a target site, in accordance with some embodiments. At a step 810, the imaging component may be inserted into the subject. At a step 820, the instrument may be inserted into the cavity towards the target site. Alternatively, the imaging component may be inserted into the cavity with the additional therapeutic and/or diagnostic instrument previously inserted into the cavity of the imaging component. At a step 830, therapy or diagnosis may be performed using the instrument at the target site. At a step 840, the instrument may be removed from the cavity.

In some embodiments, the method 800 may additionally comprise at least steps 850, 860, 870. At a step 850, the method may comprise inserting a second instrument into the cavity towards the target site. During a step 850, the imaging component may remain in situ. At a step 860, therapy or diagnosis may be performed using the second instrument at the target site. At a step 870, the second instrument may be removed from the cavity, wherein the second instrument may be different from the first instrument. In some embodiments, steps 850, 860 and 870 may be repeated using a third, forth, or more instruments.

Method 800 may represent a general method of use of an imaging component from which one of ordinary skill will recognize many variations and adaptations.

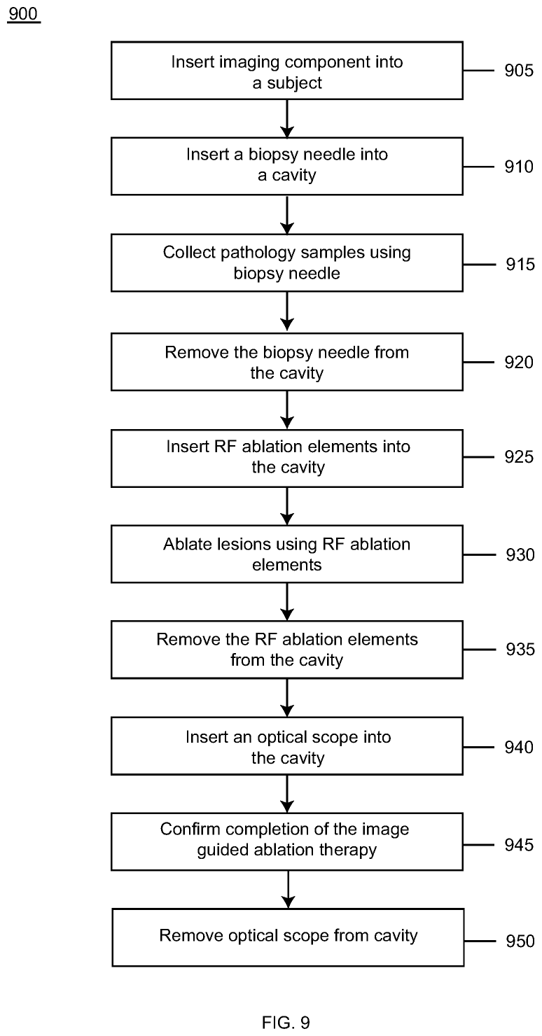

In some embodiments, the present disclosure may additionally provide a method of performing image guided ablation therapy. FIG. 9 shows an exemplary method 900 of performing image guided ablation therapy, in accordance with some embodiments. At a step 905, the imaging component may be inserted into a subject; with the imaging component in situ. At a step 910, a biopsy needle may be inserted into the cavity. At a step 915, pathology samples may be collected using the biopsy needle. At a step 920, the biopsy needle may be removed from the cavity. Test results obtained from the biopsy sample may be used to inform later steps of a method of preforming a method of image guided ablation therapy. For example, one or more biopsies and/or further imaging may inform the surgeon whether and/or where tissue needs to be excised and/or ablated, where a therapeutic and/or diagnostic agent should be delivered, and/or where addition imaging should be performed. At a step 925, radiofrequency (RF) ablation elements may be inserted into the cavity. At a step 930, lesions may be ablated using the RF ablation elements. At a step 935, the RF ablation elements may be removed from the cavity. At a step 940, an optical scope may be inserted into the cavity. At a step 945, completion of the image guided ablation therapy may be confirmed using the optical scope. At a step 950, the optical scope may be removed from the cavity. Alternatively or additionally to confirming ablation with the optical scope, the RF ablation element may be swapped out for a drug delivery device to deliver analgesic(s), hemostatic agent(s), and/or other therapeutic agents after tissue ablation or other therapeutic and/or diagnostic step.

Other exemplary methods may comprise a method of coupling instruments, comprising: advancing an imaging component to within a surgical space, wherein the imaging component comprises a shaft comprising a proximal end and a distal end; coupling a first instrument to the imaging component for use in the surgical space, wherein the first instrument may be a therapeutic or diagnostic instrument; uncoupling the first instrument from the imaging component while the imaging component remains within the surgical space; and coupling a second instrument to the imaging component for use in the surgical space while the imaging component remains within the surgical space, wherein the second instrument may be a therapeutic or diagnostic instrument different from the first instrument.

In some embodiments, a method of coupling instruments additionally comprises coupling the first instrument while the imaging component remains within the surgical space. In some embodiments, a method of coupling instruments additionally comprises coupling the first instrument while the imaging component is outside of the surgical space. In some embodiments, a method of coupling instruments additionally comprises collecting a tissue sample from the surgical space with the first instrument. In some embodiments, a method of coupling instruments additionally comprises ablating a region within the surgical space with the second instrument. In some embodiments, a method of coupling instruments additionally comprises performing therapy or diagnosis with the first instrument. In some embodiments, a method of coupling instruments additionally comprises selecting the second instrument based on data gathered from said performing therapy or diagnosis with the first instrument. In some embodiments, a method of coupling instruments additionally comprises adjusting a parameter of therapy or diagnosis performed with the second instrument based on data gathered from said performing therapy or diagnosis with the first instrument. In such embodiments, data gathered may comprise image data, and adjusting the parameter may comprise adjusting an ablation zone for the second instrument.

In another exemplary method, embodiments of the present disclosure may provide a method of performing therapy or diagnosis at a target site. A method of performing therapy may comprise: advancing an imaging component to the target site. A method of performing therapy may comprise an imaging component comprising: a shaft comprising a proximal end, a distal end, and a cavity extending across the shaft from the proximal end towards the distal end, wherein a wall of the cavity comprises an elongated opening in communication with an exterior of the shaft at least partially along the shaft. A method of performing therapy may comprise an imaging transducer coupled to the distal end of the shaft. A method of performing therapy may comprise performing therapy or diagnosis using an instrument inserted into the cavity and advanced to the target site.

In some embodiments, a method of performing therapy may additionally comprise inserting the first instrument into the cavity before advancing the imaging component to the target site. In some embodiments, a method of performing therapy may additionally comprise inserting the first instrument into the cavity after advancing the imaging component to the target site. In some embodiments, a method of performing therapy may additionally comprise removing the first instrument from the cavity while the imaging component remains at the target site. In some embodiments, a method of performing therapy may additionally comprise inserting a second instrument into the cavity and advancing the second instrument to the target site. In some embodiments, a method of performing therapy may additionally comprise performing therapy or diagnosis using the second instrument.

Methods described herein may function to perform therapy or diagnosis on a volume of tissue (e.g., a patient uterus, another organ). In some embodiments, the methods described herein may be performed in laparoscopic surgery. In such an embodiment, the methods described herein may additionally comprise insertion of a trocar into a patient bodily lumen. During laparoscopic surgery, the imaging component may be inserted into the cannula of the trocar in order to perform a surgical procedure. In some embodiments, the method may be performed non-invasively. In such embodiments, the imaging component may be inserted into a pre-existing or naturally formed patient bodily lumen. Additionally or alternatively, the method may be performed in minimally invasive surgery. In such embodiments, a lumen may be formed in a patient, which may be of a minimal size to speed healing time and minimize operative trauma.

Methods described herein may be implemented, at least in part, by way of an embodiment, variation, and/or example of the instruments and, additionally or alternatively, the imaging component 100 described herein. Additionally or alternatively, methods described herein may be implemented using any other suitable imaging component and/or instrument, and as facilitated by any of the computational and/or processing components described further below. Methods described herein may be implemented by system 500 and/or 1200. The imaging component may be used in conjunction with an instrument such as a biopsy needle; an optical scope; implantation device; therapy electrodes; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents or implants to the tissue to be treated. FIGS. 2A-2E show exemplary instruments which may be slidably received by the imaging component.

A person of ordinary skill in the art will recognize many adaptations and variations on the methods described herein. Further, one or more steps described with respect to methods herein may be deleted or repeated, additional steps may be added, and the steps may be performed in any order. Steps described with respect to one method may be added or combined with another. For example, steps of method 900 may be added to method 800.

In some embodiments, imaging components, systems, and methods described herein include a digital processing device, or use of the same. In further embodiments, the digital processing device includes one or more hardware central processing units (CPUs), general purpose graphics processing units (GPGPUs), or field programmable gate arrays (FPGAs) that carry out the device's functions. In still further embodiments, the digital processing device further comprises an operating system configured to perform executable instructions. In some embodiments, the digital processing device may be optionally connected a computer network. In further embodiments, the digital processing device is optionally connected to the internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, intern& appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle®Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

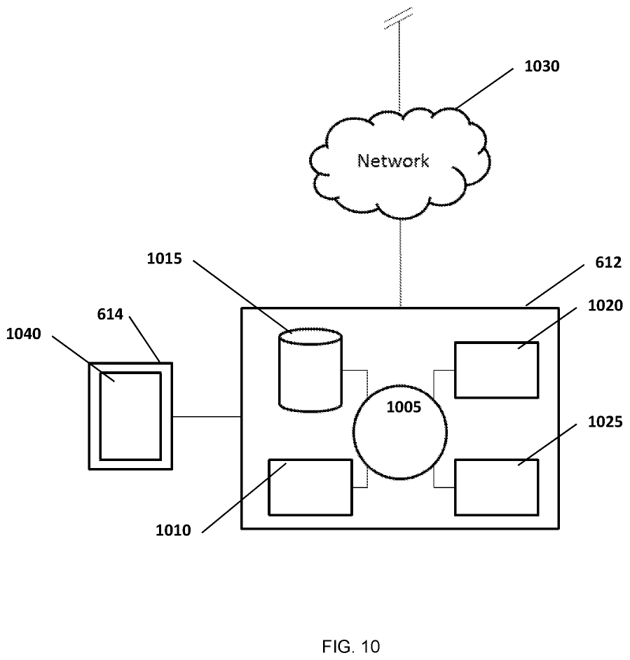

Referring to FIG. 10, in a particular embodiment, an exemplary digital processing device 612 is programmed or otherwise configured control to an imaging component and/or instruments as described herein. The device 612 may regulate various aspects of the imaging component and/or instruments of the present disclosure, such as, for example, performing processing steps. In this embodiment, the digital processing device 612 includes a central processing unit (CPU, also “processor” and “computer processor” herein) 1005, which may be a single core or multi core processor, or a plurality of processors for parallel processing. The digital processing device 612 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 may be a data storage unit (or data repository) for storing data. The digital processing device 612 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the device 612, can implement a peer-to-peer network, which may enable devices coupled to the device 612 to behave as a client or a server.

Continuing to refer to FIG. 10, the CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and write back. The CPU 1005 can be part of a circuit, such as an integrated circuit. One or more other components of the device 612 can be included in the circuit. In some cases, the circuit is an application specific integrated circuit (ASIC) or a field programmable gate array (FPGA).

Continuing to refer to FIG. 10, the storage unit 1015 can store files, such as drivers, libraries and saved programs. The storage unit 1015 can store user data, e.g., user preferences and user programs. The digital processing device 612 in some cases can include one or more additional data storage units that are external, such as located on a remote server that is in communication through an intranet or the Internet. The digital processing device 612 can communicate with one or more remote computer systems through the network 1030. For instance, the device 612 can communicate with a remote computer system of a user.

Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 612, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The digital processing device 612 can include or be in communication with an electronic display 614 that comprises a user interface (UI) 1040. Examples of UI's include, without limitation, a graphical user interface (GUI) and web-based user interface. In some cases, electronic display 614 may be connected to the computer system 612 via a network, e.g., via network 1030.

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle Basic Web, Nokia Browser, Opera Software Opera Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-121. (canceled)

122. A system for performing therapy or diagnosis at a target site within a patient, comprising:
a first therapeutic or diagnostic instrument;
a second therapeutic or diagnostic instrument different from the first therapeutic or diagnostic instrument; and
an imaging component configured to be removably coupled to both the first and second therapeutic or diagnostic instruments, either simultaneously or individually;
wherein the imaging component is configured to be deliverable to the target site within the patient both (i) separately from the first and second therapeutic or diagnostic instruments, and (ii) coupled with the first or second therapeutic or diagnostic instruments, and
wherein the imaging component is configured to be removably coupled to both the first and second therapeutic or diagnostic instruments, either simultaneously or individually, after the imaging component is delivered to the target site within the patient.

123. The system of claim 122, wherein the first and second therapeutic or diagnostic instruments comprise two of the following: a tissue collector, a tissue ablation element, an optical scope, or therapy electrodes.

124. The system of claim 123, wherein the tissue collector comprises a biopsy needle.

125. The system of claim 123, wherein the tissue ablation element comprises one or more of a radiofrequency (RF) ablation element, an ultrasonic ablation element, a heat-based ablation element, or a cryoablation element.

126. The system of claim 122, wherein the imaging component comprises a shaft comprising a proximal end, a distal end, and a cavity extending across the shaft from the proximal end towards the distal end, wherein a wall of the cavity comprises an elongated opening in communication with an exterior of the shaft at least partially along the shaft.

127. The system of claim 126, wherein the cavity is defined by an exterior surface of the shaft.

128. The system of claim 127, wherein the exterior surface of the shaft comprises only atraumatic edges.

129. The system of claim 126, wherein an edge of the elongated opening is bent towards an interior of the cavity.

130. The system of claim 126, wherein the cavity is configured to slidably receive the instrument.

131. The system of claim 126, wherein a distal portion of the cavity is angled axially relative to the shaft.

132. The system of claim 131, wherein the distal portion of the cavity is angled at about 3 to 45 degrees axially relative to the shaft.

133. The system of claim 126, further comprising a tube.

134. The system of claim 133, wherein the tube is aligned to be in parallel with the shaft of the imaging component.

135. The system of claim 134, wherein the tube is rotatable relative to the shaft while the shaft remains stationary.

136. The system of claim 133, wherein the tube comprises a lumen configured to slidably receive the first or second instrument.

137. The system of claim 136, wherein the tube is configured to slidably receive the first or second instrument after the first or second instrument is aligned to be in parallel with the shaft of the imaging component.

138. The system of claim 137, wherein the first or second instrument is rotatable relative to the shaft while the shaft remains stationary.

139. The system of claim 133, wherein the tube is disposable.

140. The system of claim 126, wherein the shaft is flexible.

141. The system of claim 140, wherein the shaft is controllably flexed along its longitudinal axis via a flex mechanism.

142. The system of claim 122, wherein the imaging component comprises an imaging transducer comprising a light emitting diode (LED) or a camera.

143. The system of claim 126, wherein the cavity defines a circular cross sectional area.

144. The system of claim 126, wherein the cavity comprises a substantially uniform cross sectional area along the shaft.

145. The system of claim 126, wherein the cavity comprises an asymmetrical cross sectional area.

146. The system of claim 126, wherein the cavity extends across the shaft from the proximal end to the distal end.

147. The system of claim 142, wherein the imaging transducer comprises an ultrasound transducer.

148.-180. (canceled)
